(19) United States
(12) Patent Application Publication
(10) Pub. No.: US 2019/0223948 A1
(43) Pub. Date: Jul. 25, 2019

(54) ENERGY DELIVERY RETURN PATH DEVICES AND METHODS

(71) Applicant: Medtronic, Inc., Minneapolis, MN (US)

(72) Inventors: Mark T. STEWART, Lino Lakes, MN (US); Brian T. HOWARD, Hugo, MN (US)

(21) Appl. No.: 16/251,807

(22) Filed: Jan. 18, 2019

Related U.S. Application Data
(60) Provisional application No. 62/620,189, filed on Jan. 22, 2018.

(51) Int. Cl. A61B 18/14; A61B 18/12

(52) U.S. Cl. CPC A61B 18/1492; A61B 2018/00577; A61B 18/1206

(57) ABSTRACT

A device, system, and method for ablating tissue with pulsed field ablation energy while minimizing stimulation of skeletal muscle and nerves, as well as minimizing damage to non-targeted tissue. Some embodiments provide a device, system, and method for delivering pulsed field ablation energy to tissue from at least one energy delivery electrode on an energy delivery device to at least one energy return electrode, which may be located on the energy delivery device and/or on a sheath or secondary device. The at least one energy delivery electrode has a surface area for the application of energy that is smaller than the surface area for the receipt or return of energy of the at least one energy return electrode.

### CROSS-REFERENCE TO RELATED APPLICATION

This application is related to and claims priority to U.S. Provisional Patent Application Ser. No. 62/620189, filed Jan. 22, 2018, entitled ENERGY DELIVERY RETURN PATH DEVICES AND METHODS, the entirety of which is incorporated herein by reference.

### FIELD

This disclosure relates to a device, system, and method for ablating tissue with pulsed field ablation energy while minimizing stimulation of skeletal muscle and nerves, as well as minimizing damage to non-targeted tissue. Specifically, some embodiments provide a device, system, and method for delivering pulsed field ablation energy to tissue from at least one energy delivery electrode on an energy delivery device to at least one energy return electrode, which may be located on the energy delivery device and/or on a sheath or secondary device. The at least one energy delivery electrode has a surface area for the application of energy that is smaller than the surface area for the receipt or return of energy of the at least one energy return electrode.

### BACKGROUND

Tissue ablation is a medical procedure commonly used to treat conditions such as cardiac arrhythmia, which includes atrial fibrillation. For treating cardiac arrhythmia, ablation can be performed to modify tissue, such as to stop aberrant electrical propagation and/or disrupt aberrant electrical conduction through cardiac tissue. The most commonly used thermal ablation technique is delivery of radiofrequency (RF) energy to heat the targeted tissue to produce lesions by coagulative necrosis. Most commonly, such RF energies are delivered focally from a catheter tip electrode in a unipolar fashion to an energy return electrode, such as a patch on the patient's skin surface. As long as the skin patch surface area is great enough, no thermal injury will be produced in the body near the return electrode. Additionally, the 500 KHz RF energy generally does not produce skeletal muscle stimulation or patient discomfort. Although thermal ablation techniques are frequency used, such as cryoablation and radiofrequency (RF) ablation, non-thermal techniques such as pulsed field ablation (PFA) may also be used.

Pulsed field ablation involves the application of short pulsed electric fields (PEF), which may reversibly or irreversibly destabilize cell membranes through electropermeablization but generally do not affect the structural integrity of the tissue components, including the acellular cardiac extracellular matrix. Pulsed field ablation may entail more than one pulse of, for example, approximately 1-100 microseconds in pulse width delivered in a train or series of monophasic or biphasic pulses with a delay of 1-1000 microseconds between each pulse. The nature of PFA allows for very brief periods of therapeutic energy delivery, on the order of tens of milliseconds in duration. Further, PFA may not cause collateral damage to non-targeted tissue as frequently or as severely as thermal ablation techniques. Additionally, pharmacologic or therapeutic agents may be preferentially introduced into the cells of targeted tissue that are exposed to PEF having reversible membrane permeablization. The energy applied may also be chosen to cause only, or predominantly, reversible cellular permeabilization effects for such delivery of therapeutic agents.

Although PFA is a relatively safe way of delivering ablation energy, it is still important to deliver the pulsed energy precisely to the target tissue area and to avoid unnecessarily exposing non-targeted structures to electric field gradients that exceed the threshold for cell death. Also important in an ablation procedure is the avoidance of stimulation of major skeletal muscle groups or nerves that would cause bodily motion and patient discomfort. While the short pulse durations used in PFA deliveries might be expected to minimize muscle stimulation, the high voltages applied to these short pulses may result in stimulation of skeletal muscle when sufficient electric field gradients are in close proximity to muscle tissue or nerves. Such stimulation may cause the patient to move excessively during the ablation procedure, which can result in undesired repositioning of the ablation device and/or alteration of the energy return path. Further, all intracardiac stimulation, recording, and ablation catheters are affected by cardiac motion, respiratory motion, device stiffness/maneuverability, and random patient movements. These sources of motion affect the positional stability and quality of electrode contact with, for example, the heart wall, and can affect the path of energy delivery, which may impair the effectiveness of the treatment. During energy delivery to ablate the target tissue, this motion can reduce effectiveness of such deliveries during the periods when the electrodes move away from the target tissue.

### SUMMARY

Some embodiments advantageously provide a method and system for ablating tissue with pulsed field ablation energy while minimizing the stimulation of or damage to non-targeted tissue. Specifically, some embodiments provide a device, system, and method for delivering pulsed field ablation energy to tissue from at least one energy delivery electrode on an energy delivery device to at least one energy return electrode, which may be located on the energy delivery device and/or on a sheath or secondary device. The at least one energy delivery electrode has a surface area for the application of energy that is generally smaller than the surface area for the receipt or return of energy of the at least one energy return electrode. The user may find it desirable to choose the relative electrode surface areas between which to deliver energy. If the user desires to produce a transmural ablation through thick tissue, an electrode area of similar or identical size may be selected to be placed on either side of the targeted tissue. For example, an energy delivery electrode area may be placed on the endocardial surface and an energy return electrode area may be placed on the epicardial surface. Such a configuration will promote transmural lesion formation. If the user desires to ablate from the endocardial aspect, non-transmurally, it may be advantageous to use a smaller energy delivery electrode area on the endocardial side while using a larger energy return electrode area on the epicardial side. Such a configuration will promote lesion formation from the endocardial aspect and ending in the mid-myocardium, while only a superficial lesion may be formed on the epicardium.

In one embodiment, a system for ablating tissue comprises: at least one energy delivery electrode having a first surface area; an energy generator in electrical communication with the at least one energy delivery electrode and being configured to transmit an electrical current to the at least one energy delivery electrode; and at least one energy return electrode having a second surface area that is greater than the first surface area, the at least one energy return electrode being in electrical communication with the at least one energy delivery electrode, such that electrical current delivered from the at least one energy delivery electrode to an area of tissue flows to the at least one energy return electrode.

In one aspect of the embodiment, the system further comprises an energy delivery device, the at least one energy delivery electrode being on the energy delivery device.

In one aspect of the embodiment, the energy delivery device includes an elongate body having a distal portion, the at least one energy return electrode being on the distal portion of the elongate body at a location that is proximal to the at least one energy delivery electrode.

In one aspect of the embodiment, the at least one energy return electrode includes a plurality of electrodes that each extends at least partially around a circumference of the elongate body. In one aspect of the embodiment, the distal portion of the elongate body includes a distal tip and the at least one energy delivery electrode is an energy delivery electrode located at the distal tip. In one aspect of the embodiment, the energy delivery electrode is a needle-shaped electrode.

In one aspect of the embodiment, the system further comprises a sheath, the energy delivery device being longitudinally movable within the sheath, the at least one energy return electrode being on the sheath and the at least one energy return electrode being movable relative to the at least one energy delivery electrode.

In one aspect of the embodiment, the system further comprises a secondary device, the at least one energy return electrode being on the secondary device.

In one aspect of the embodiment, the secondary device includes an expandable element having a conductive mesh.

In one aspect of the embodiment, the secondary device includes: a secondary device elongate body having a distal portion; an expandable element coupled to a first side of the distal portion of the secondary device elongate body; and a conductive portion coupled to a second side of the distal portion of the secondary device elongate body, the second side being opposite the first side, the conductive portion including the at least one energy return electrode.

In one aspect of the embodiment, the at least one energy return electrode includes a plurality of electrodes and the secondary device includes a secondary device elongate body having a distal portion, the distal portion being transitionable between a linear first configuration and a spiral-shaped second configuration, the plurality of energy return electrodes being on a first side of the distal portion such that the plurality of energy return electrodes are coplanar with the distal portion is in the spiral-shaped second configuration.

In one aspect of the embodiment, the secondary device includes: a secondary device elongate body having a distal portion, the distal portion being transitionable between a linear first configuration and a spiral-shaped second configuration, the distal portion including a plurality of apertures; and an electrically conductive conductor insertable or translatable into the secondary device elongate body such that at least a portion of the conductor is exposed through the plurality of apertures, the at least one energy return electrode being the at least a portion of the conductor that is exposed through the plurality of apertures. In one aspect of the embodiment, the apertures are radially arranged about the distal portion of the secondary device elongate body. In one aspect of the embodiment, the secondary device elongate body has a tissue-contacting surface when the secondary device elongate body is in the spiral-shaped second configuration, the plurality of apertures being on the tissue-contacting surface.

In one aspect of the embodiment, the secondary device includes: a secondary device elongate body having a distal portion, the distal portion being transitionable between a first configuration and an expanded second configuration; a shaft at least partially within the secondary device elongate body, the shaft including a distal portion and a longitudinal axis; an expandable element that is coupled to the distal portion of the shaft, the expandable element being at least partially wound about the shaft, the expandable element having an electrically conductive first surface and an electrically insulated second surface opposite the first surface, rotation of the shaft about its longitudinal axis causing the expandable element to transition between a first configuration and an expanded second configuration. In one aspect of the embodiment, the expandable element is a sheet.

In one embodiment, a method for ablating an area of tissue using pulsed field ablation energy comprises: positioning at least one energy delivery electrode at a first location proximate the area of tissue, the at least one energy delivery electrode having a first surface area; positioning at least one energy return electrode at a second location different than the first location, the at least one energy return electrode having a second surface area that is greater than the first surface area; and delivering the pulsed field ablation energy from the at least one energy delivery electrode to the area of tissue, such that the pulsed field ablation energy flows from the area of tissue to the at least one energy return electrode.

In one aspect of the embodiment, the at least one energy delivery electrode is on an energy delivery device and the at least one energy return electrode is on secondary device.

In one aspect of the embodiment, the first location is an endocardial location and the second location is an endocardial location. In one aspect of the embodiment, the first location is one of within a cardiac vein, within a cardiac artery, in contact with tissue surrounding a pulmonary vein ostium, within a superior vena cava, within an inferior vena cava, within an atrial appendage, within a right atrium, within a left atrium, within a right ventricle, within a left ventricle, within a coronary sinus, within an aorta, within a pulmonary artery, and within a pulmonary vein; and the second location is one of within a cardiac vein, within a cardiac artery, in contact with tissue surrounding a pulmonary vein ostium, within a superior vena cava, within an inferior vena cava, within an atrial appendage, within a right atrium, within a left atrium, within a right ventricle, within a left ventricle, within a coronary sinus, within an aorta, within a pulmonary artery, and within a pulmonary vein.

In one aspect of the embodiment, the first location is an endocardial location and the second location is an epicardial location. In one aspect of the embodiment, the first location is one of within a cardiac vein, within a cardiac artery, in contact with tissue surrounding a pulmonary vein ostium, within a superior vena cava, within an inferior vena cava, within an atrial appendage, within a right atrium, within a left atrium, within a right ventricle, within a left ventricle, within a coronary sinus, within an aorta, within a pulmonary artery, and within a pulmonary vein; and the second location is one of within a pericardial space, at a location outside but adjacent a pericardium, in contact with atrial epicardial tissue, and in contact with ventricular epicardial tissue.

In one aspect of the embodiment, the first location is an epicardial location and the second location is an endocardial location. In one aspect of the embodiment, the first location is one of within a pericardial space, at a location outside but adjacent a pericardium, in contact with atrial epicardial tissue, and in contact with ventricular epicardial tissue; and the second location is one of within a cardiac vein, within a cardiac artery, in contact with tissue surrounding a pulmonary vein ostium, within a superior vena cava, within an inferior vena cava, within an atrial appendage, within a right atrium, within a left atrium, within a right ventricle, within a left ventricle, within a coronary sinus, within an aorta, within a pulmonary artery, and within a pulmonary vein.

In one embodiment, a system for ablating tissue comprises: a first device having a plurality of first electrodes, each of the plurality of first electrodes being independently operable; a second device having a plurality of second electrodes, each of the plurality of second electrodes being independently operable; and an energy generator in electrical communication with the plurality of first electrodes and the plurality of second electrodes and being configured to selectively transmit an electrical current to each electrode of the plurality of first plurality of electrodes and to each electrode of the plurality of second electrodes, the first device transmitting energy from the energy generator to the second device when a first number of electrodes of the plurality of first electrodes is activated that is less than a second number of electrodes of the plurality of second electrodes, and the second device transmitting energy from the energy generator to the first device when the first number of electrodes of the plurality of first electrodes is activated that is greater than a second number of electrodes of the plurality of second electrodes.

### BRIEF DESCRIPTION OF THE DRAWINGS

A more complete understanding of embodiments described herein, and the attendant advantages and features thereof, will be more readily understood by reference to the following detailed description when considered in conjunction with the accompanying drawings wherein:

FIG. 1 shows an exemplary medical system, the system including an exemplary embodiment of an energy delivery device and an exemplary embodiment of a sheath having at least one energy return electrode in accordance with the present disclosure;

FIG. 2 shows another exemplary medical system, the system including an exemplary embodiment of an energy delivery device having at least one energy return electrode in accordance with the present disclosure;

FIG. 3 shows another exemplary medical system, the system including an exemplary energy delivery device and an exemplary embodiment of a secondary device having at least one energy return electrode in accordance with the present disclosure;

FIG. 4 shows another exemplary embodiment of a sheath having at least one energy return electrode in accordance with the present disclosure;

FIG. 5 shows another exemplary embodiment of a sheath having at least one energy return electrode in accordance with the present disclosure;

FIG. 6 shows another exemplary embodiment of a sheath having at least one energy return electrode in accordance with the present disclosure;

FIG. 7 shows another exemplary embodiment of a sheath having at least one energy return electrode in accordance with the present disclosure;

FIG. 8 shows a flattened view of the energy return electrodes of FIG. 7 in accordance with the present disclosure, the energy return electrodes being separated from the sheath for clarity;

FIG. 9 shows another exemplary embodiment of a sheath having at least one energy return electrode in accordance with the present disclosure;

FIG. 10 shows another exemplary embodiment of an energy delivery device having at least one return electrode in accordance with the present disclosure;

FIG. 11 shows another exemplary embodiment of an energy delivery device having at least one return electrode in accordance with the present disclosure;

FIG. 12 shows another exemplary embodiment of an energy delivery device having at least one return electrode in accordance with the present disclosure;

FIG. 13 shows another exemplary embodiment of an energy delivery device having at least one return electrode in accordance with the present disclosure;

FIG. 14 shows another exemplary embodiment of an energy delivery device having at least one return electrode in accordance with the present disclosure;

FIG. 15 shows another exemplary embodiment of an energy delivery device in accordance with the present disclosure;

FIG. 16 shows another exemplary embodiment of an energy delivery device in accordance with the present disclosure;

FIG. 17 shows another exemplary embodiment of an energy delivery device in accordance with the present disclosure;

FIG. 18 shows a side view of another exemplary embodiment of a secondary device having at least one energy return electrode in accordance with the present disclosure;

FIG. 19 shows a front view of the secondary device of FIG. 18 in accordance with the present disclosure;

FIG. 20 shows a side view of another exemplary embodiment of a secondary device having at least one energy return electrode in accordance with the present disclosure;

FIG. 21 shows a front view of the secondary device of FIG. 20 in accordance with the present disclosure;

FIG. 22 shows a side view of another exemplary embodiment of a secondary device having at least one energy return electrode in accordance with the present disclosure;

FIG. 23 shows a front view of the secondary device of FIG. 22 in accordance with the present disclosure;

FIG. 24 shows a side view of another exemplary embodiment of a secondary device having at least one energy return electrode, the secondary device being in an uninflated configuration in accordance with the present disclosure;

FIG. 25 shows a front view of the secondary device of FIG. 24 in accordance with the present disclosure;

FIG. 26 shows a side view of the secondary device of FIG. 24 in an inflated configuration in accordance with the present disclosure;

FIG. 27 shows a front view of the secondary device of FIG. 26 in accordance with the present disclosure;

FIG. 28 shows an exemplary energy delivery device positioned on a first side of an area of target tissue and a secondary device having at least one energy return electrode positioned on a second side of the area of target tissue in accordance with the present disclosure;

FIG. 29 shows another exemplary embodiment of a secondary device having at least one energy return electrode in accordance with the present disclosure;

FIG. 30 shows a first (non-tissue-contacting) surface of a secondary device in accordance with the present disclosure;

FIG. 31 shows a second (tissue-contacting) surface of another exemplary embodiment of a secondary device and an insertable energy return stylet in accordance with the present disclosure;

FIG. 32 shows a second (tissue-contacting) surface of the exemplary embodiment of a secondary device of FIG. 31, the insertable energy return stylet being within the secondary device in accordance with the present disclosure;

FIG. 33 shows another exemplary embodiment of a secondary device having at least one energy return electrode, the secondary device being in a first configuration in accordance with the present disclosure;

FIG. 34 shows the secondary device of FIG. 32, the secondary device being in an expanded configuration in accordance with the present disclosure;

FIG. 35 shows another exemplary embodiment of a secondary device having at least one energy return electrode in accordance with the present disclosure;

FIG. 36 shows a front view of another exemplary embodiment of a secondary device having at least one energy return electrode in accordance with the present disclosure;

FIG. 37 shows a side view of the secondary device of FIG. 36 in accordance with the present disclosure;

FIG. 38 shows a front view of another exemplary embodiment of a secondary device having at least one energy return or delivery electrode in accordance with the present disclosure;

FIG. 39 shows a rear view of the secondary device shown in FIG. 38 in accordance with the present disclosure;

FIG. 40 shows another exemplary embodiment of a secondary device having at least one energy return electrode in accordance with the present disclosure;

FIG. 41 shows an exemplary placement of a secondary device within a heart in accordance with the present disclosure;

FIG. 42 shows an exemplary placement of an energy delivery device on an epicardial surface of the heart, relative to the placement of the secondary device shown in FIG. 41 in accordance with the present disclosure;

FIG. 43 shows another exemplary placement of an energy delivery device within a heart in accordance with the present disclosure;

FIG. 44 shows another exemplary placement of a secondary device on an epicardial surface of the heart, relative to the placement of the energy delivery device shown in FIG. 43 in accordance with the present disclosure;

FIG. 45 shows another exemplary placement of a secondary device on an epicardial surface of the heart, relative to the placement of the energy delivery device shown in FIG. 43 in accordance with the present disclosure;

FIG. 46 shows an exemplary placement of an energy delivery device within the right pulmonary artery of a heart, the energy delivery device having at least one energy return electrode in accordance with the present disclosure;

FIG. 47 shows an exemplary placement of the energy delivery device of FIG. 46 within the left pulmonary artery of the heart in accordance with the present disclosure;

FIG. 48 shows another exemplary placement of an energy delivery device within the right pulmonary artery of a heart, the energy delivery device having at least one energy return electrode in accordance with the present disclosure; and

FIG. 49 shows another exemplary placement of the energy delivery device of FIG. 48 within the left pulmonary artery in accordance with the present disclosure.

### DETAILED DESCRIPTION

Before describing in detail exemplary embodiments, it is noted that the embodiments reside primarily in combinations of apparatus components and processing steps related to delivering ablation energy, such as pulsed field ablation energy, between an energy delivery electrode and an energy return electrode, while minimizing collateral damage to non-target tissues. Accordingly, the system and method components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

As used herein, relational terms, such as “first” and “second,” “top” and “bottom,” and the like, may be used solely to distinguish one entity or element from another entity or element without necessarily requiring or implying any physical or logical relationship or order between such entities or elements. The terminology used herein is for the purpose of describing particular embodiments only and is not intended to be limiting of the concepts described herein. As used herein, the singular forms “a”, “an” and “the” are intended to include the plural forms as well, unless the context clearly indicates otherwise. It will be further understood that the terms “comprises,” “comprising,” “includes” and/or “including” when used herein, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this disclosure belongs. It will be further understood that terms used herein should be interpreted as having a meaning that is consistent with their meaning in the context of this specification and the relevant art and will not be interpreted in an idealized or overly formal sense unless expressly so defined herein.

In embodiments described herein, the joining term, “in communication with” and the like, may be used to indicate electrical or data communication, which may be accomplished by physical contact, induction, electromagnetic radiation, radio signaling, infrared signaling or optical signaling, for example. One having ordinary skill in the art will appreciate that multiple components may interoperate and modifications and variations are possible of achieving the electrical and data communication.

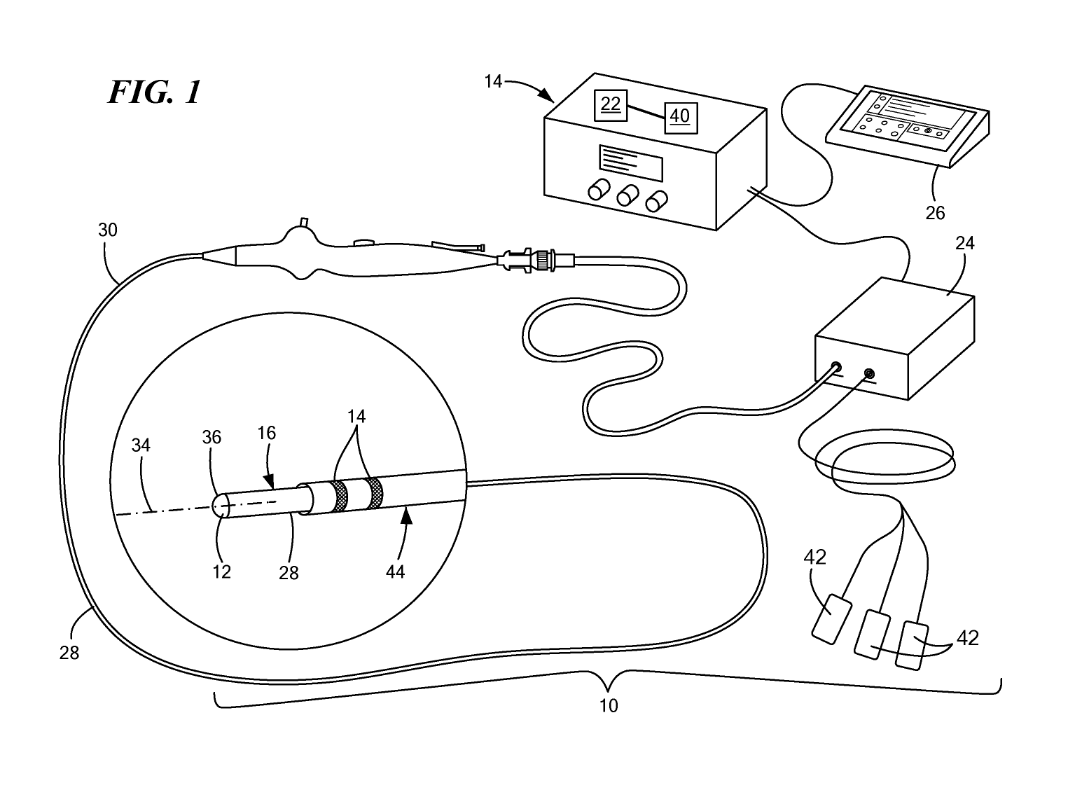

FIGS. 1-3 generally show exemplary medical systems 10, in which the medical systems 10 generally include at least one energy delivery electrode 12 and at least one energy return electrode 14. In general, each medical system 10 includes at least an energy delivery device 16 in communication with a control unit 18, and at least one energy return electrode 14. Referring now to FIG. 1, in one embodiment the energy delivery device 16 includes the at least one energy delivery electrode 12 and the control unit 18 includes an energy generator 22 configured to transmit energy, such as pulsed field ablation energy, to the at least one energy delivery electrode 12. Although the systems and devices disclosed herein are described as being used for the delivery of pulsed field ablation energy (for example, to cause irreversible electroporation of tissue), it will be understood that other energy modalities may be used in addition to or instead of pulsed field ablation. For example, the energy delivery device 16 may also be used to ablate tissue through cryoablation, laser ablation, microwave ablation, radiofrequency (RF) ablation, and the like.

The energy delivery device 16 may be coupled directly to the energy generator 22 and the energy generator 22 may include an energy control, delivering, and monitoring system. Alternatively, the energy delivery device 16 may be coupled indirectly to the energy generator 22 through a device electrode distribution system 24 (which may also be referred to herein as a catheter electrode distribution system or CEDS). In one embodiment, the medical system 10 also includes a remote controller 26 that is in communication with the energy generator 22 for operating and controlling the various functions of the energy generator 22.

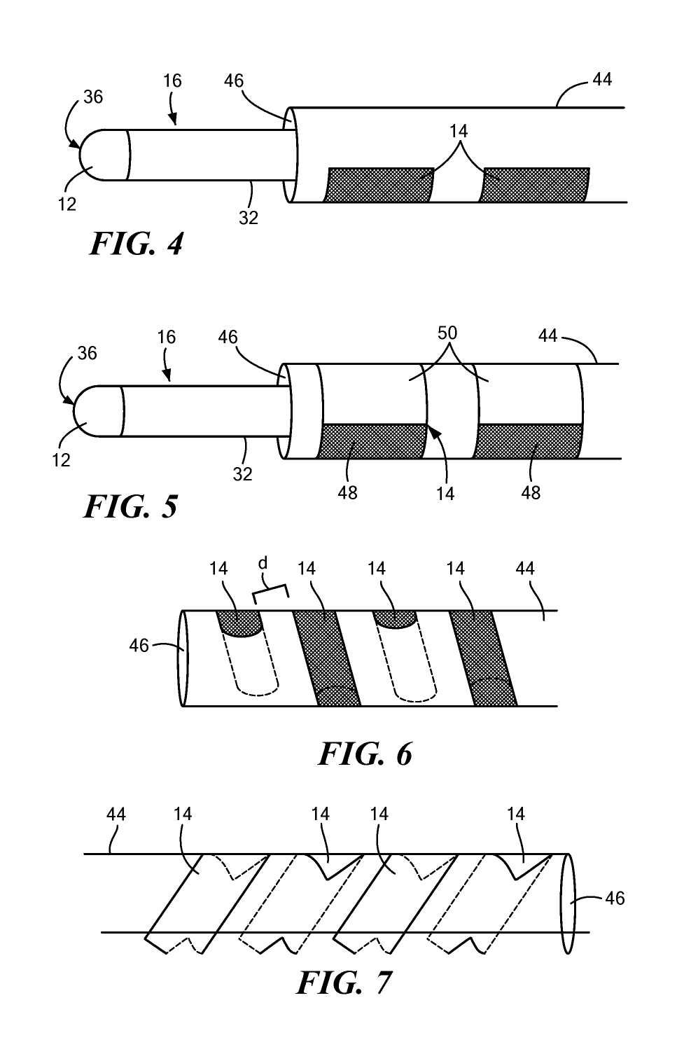

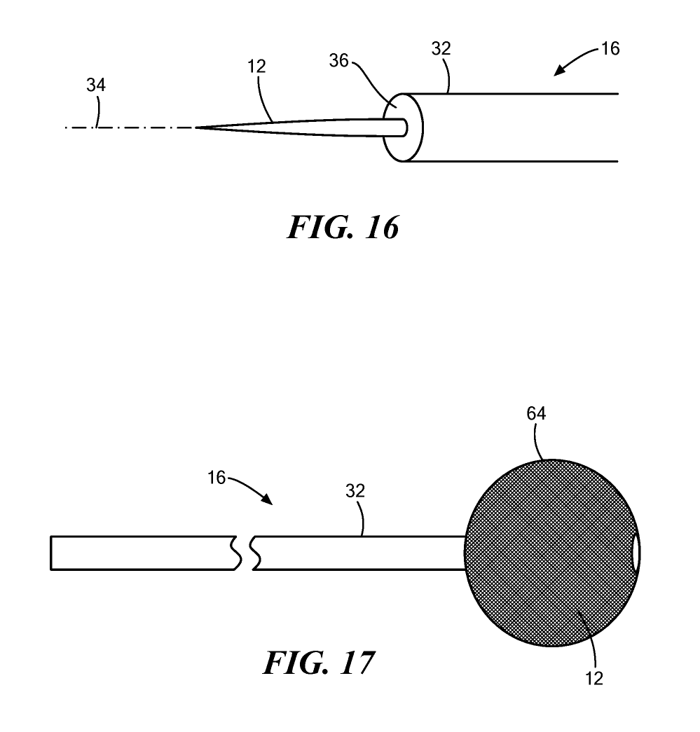

In one embodiment, the energy delivery device 16 includes an elongate body 28 passable through a patient's vasculature and/or proximate to a tissue region for diagnosis and/or treatment. For example, the energy delivery device 16 may be a catheter that is deliverable to the tissue region via a sheath or intravascular introducer (as shown in FIG. 1). As is shown in more detail in FIGS. 4 and 5, the elongate body 28 defines a proximal portion 30, a distal portion 32, and a longitudinal axis 34, and may further include one or more lumens disposed within the elongate body 28 to provide mechanical, electrical, and/or fluid communication between the proximal portion 30 and the distal portion 32 of the elongate body 28. In one embodiment, the energy delivery device 16 is focal device that includes an energy delivery electrode 12 in the distal portion 32 of the elongate body 28, such as at the distal tip 36 of the elongate body 28 (for example, as shown in FIG. 1). In one embodiment, the energy delivery device 16 includes an energy delivery electrode 12 at the distal tip 36 of the elongate body 28 and mapping electrodes 38 proximal to the energy delivery electrode 12 (for example, mapping electrodes 38 are shown in FIG. 15). In one embodiment, the energy delivery device 16 includes a needle-shaped energy delivery electrode 12 configured to be at least partially inserted into tissue (for example, as shown in FIG. 16). For example, the energy delivery device 16 of FIG. 16 may be used to create lesions deep within the tissue.

As is discussed below, in one embodiment the energy delivery device 16 serves both as a treatment device and a mapping device. The at least one energy delivery electrode 12 may not only deliver ablation energy, but may also perform diagnostic functions, such as the collection of intracardiac electrograms (EGMs) and/or monophasic action potentials (MAPs), as well as performing selective pacing of intracardiac sites for diagnostic purposes. Additionally or alternatively, the energy delivery device 16 may include one or more mapping electrodes 38 (for example, as shown in FIG. 15). Measured or recorded signals may then be transferred from the energy delivery device 16 through the CEDS 24 (if the system includes the CEDS) to a recording system input box, which may be included or integrated with the energy generator 22. If a CEDS 24 is used, the energy delivery electrode(s) 12 may also monitor the proximity to target tissues and quality of contact with such tissues using impedance-based measurements with connections to the CEDS 24. In one embodiment, the CEDS 24 includes high-speed relays to disconnect/reconnect specific energy delivery electrode(s) 12 from the energy generator 22 during an energy delivery procedure. Immediately following the pulsed energy deliveries, the relays reconnect the energy delivery electrode(s) 12 so they may be used for diagnostic purposes.

Although not shown, the medical system 10 may include one or more sensors to monitor the operating parameters throughout the system, in addition to monitoring, recording, or otherwise conveying measurements or conditions within the energy delivery device 16, ambient environment at the distal portion 32 of the energy delivery device 16, and/or one or more secondary devices or system components. The sensor(s) may be in communication with the energy generator 22 and/or the CEDS 24 for initiating or triggering one or more alerts or energy delivery modifications during operation of the medical system.

In one embodiment, the energy generator 22 includes processing circuitry 40 having a processor in communication with one or more controllers and/or memories containing software modules with instructions or algorithms to provide for the automated operation and performance of the features, sequences, calculations, or procedures described herein and/or required for a given medical procedure. In one embodiment, the medical system 10 further includes a plurality of surface ECG electrodes 42 in electrical communication with the energy generator 22 through the CEDS 24. The plurality of surface ECG electrodes 42 may be part of a positioning and navigation system that allows for the localization of the electrodes within three-dimensional space within the patient's body through the transmission and receipt of positioning and navigation signals to and from the energy generator 22. When the surface ECG electrodes 42 are applied to the skin of a patient, they may be used, for example, to monitor the patient's cardiac activity to determine pulse train delivery timing at the desired portion of the cardiac cycle (that is, to record and transmit electrical activity measurements to the generator) and/or for navigation and location of the energy delivery device 16 within the patient. In addition to monitoring, recording, or otherwise conveying measurements energy delivery device 16, additional measurements may be made, such as temperature, electrode-tissue interface impedance, delivered charge, current, power, voltage, work, or the like in the energy generator 22 and/or the energy delivery device 16. In one embodiment, the surface ECG electrodes 42 are be in communication with the energy generator 22 for determining the timing during a cardiac cycle at which to initiate or trigger one or more alerts or therapeutic deliveries during operation of the energy delivery device 16. Additional neutral electrode patient ground patches (not shown) may be used to evaluate the desired bipolar electrical path impedance, as well as monitor and alert the user upon detection of undesired and/or unsafe conditions.

The energy generator 22 provides electrical pulses to the energy delivery device 16 to perform an electroporation procedure to cardiac tissue or other tissues within the patient's body, such as renal tissue, airway tissue, and organs or tissue within the cardiac space. Specifically, in one embodiment the energy generator 22 is be configured and programmed to deliver pulsed, high-voltage electric fields appropriate for achieving desired pulsed, high -voltage ablation (referred to as “pulsed field ablation” or “pulsed electric field ablation”) and/or pulsed radiofrequency ablation. The pulse trains delivered by the energy generator 22 may be delivered at a frequency less than 30 kHz, and in an exemplary configuration, 1 kHz, which is a lower frequency than radiofrequency treatments. The pulsed-field energy in accordance with the present disclosure may be sufficient to induce cell death for purposes of completely blocking an aberrant conductive pathway along or through cardiac tissue, destroying the ability of the so-ablated cardiac tissue to propagate or conduct cardiac depolarization waveforms and associated electrical signals. Additionally or alternatively, the energy generator 22 may be configured and programmed to deliver RF energy appropriate for achieving tissue ablation.

The at least one energy delivery electrode 12 has a smaller surface area, or energy delivery surface area, than the surface area, or energy return surface area, of the at least one energy return electrode 14. Delivering pulsed field ablation energy from a smaller surface area (which may be referred to as delivery the energy focally) may avoid stimulation that might occur if the delivery used a ground skin patch return path. Also, delivering pulsed field ablation energy from a small surface area minimizes the electric current, which reduces or avoids the delivery of an excess amount of energy that would be ineffective and wasted in the blood pool. If the energy delivery device 16 includes a plurality of energy delivery electrodes 12, a series of different bipolar energy vectors may be used between the plurality of energy delivery electrodes 12, followed by or proceeded by energy delivery between one or more of the plurality of energy delivery electrodes 12 and the at least one ground electrode. When pulsed field ablation energy is delivered between the energy delivery electrode(s) 12 and the energy return electrode(s) 14, the electrical current is transmitted from the energy generator 22 to the at least one energy delivery electrode 12, and is then delivered from the at least one energy delivery electrode 12 to the tissue (for example, an area of target tissue). The electrical current then flows from the at least one energy delivery electrode 12 to the at least one energy return electrode 14, due to the larger surface area of the at least one energy return electrode 14. Energy delivery between the energy delivery electrode(s) 12 and the energy return electrode(s) 14 is considered to be unipolar energy delivery because the electrode area of the energy return electrode(s) 14 is greater than the electrode area of the energy delivery electrode(s) 12. The ablative effect may be controlled through the relative positioning of the energy return electrode(s) 14 and the energy delivery electrode(s) 12. In one non-limiting example, the energy return electrode(s) 14 may be positioned within the right or left ventricular outflow tracts, such as within the pulmonary arterial system or within the aorta. Additionally or alternatively, the energy delivery electrode(s) 12 may be positioned within the coronary sinus or great coronary vein of the heart. Field vectors from such locations may promote the creation of transmural lesions through the left atrial wall in such areas. Further, delivery of energy in this unipolar mode may be combined with delivery of energy in bipolar mode (that is, between the energy delivery electrode(s) 12 of the energy delivery device 16).

Referring now to FIG. 1, the medical system 10 includes an energy delivery device 16 and a sheath 44 having at least one energy return electrode 14. The sheath 44 may be used for navigation and placement of the energy delivery at the treatment site, as well as to create an energy return vector from the energy delivery device 16. The sheath 44 includes a lumen 46 that is sized and configured to receive the energy delivery device 16 therein, and the energy delivery device 16 and the sheath 44 are longitudinally movable relative to each other. The lumen 46 is shown in FIGS. 4-7 and 9. Movement of the sheath 44 relative to the energy delivery device 16 allows adjustment of the position of the at least one energy return electrode 14 and, consequently, adjustment of the energy vector during delivery of pulsed field ablation energy.

Referring now to FIGS. 4-9, additional embodiments of the sheath 44 shown in FIG. 1 are shown. In some embodiments, the portion of the sheath 44 that includes energy return electrodes 14 is tens of centimeters long. Referring to FIG. 4, in one embodiment the sheath 44 includes a plurality of energy return electrodes 14 that each extend around a portion (that is, less than an entirety) of the circumference of the sheath 44. In this embodiment, the entirety of each energy return electrode 14 is conductive. Referring to FIG. 5, in another embodiment the sheath 44 includes a plurality of energy return electrodes 14 that extend around an entirety of the circumference of the sheath 44, and each energy return electrode 14 has a conductive portion 48 and a non-conductive portion 50. Thus, the conductive portion 48 of each energy return electrode 14 extends around only a portion (that is, less than an entirety) of the circumference of the sheath 44. In one embodiment, conductive portion 48 is composed of a conductive first material and the non-conductive portion 50 is composed of a non-conductive second material. Additionally or alternatively, the non-conductive portion 50 is composed of a conductive material, but includes a non-conductive coating and/or oxide of the conductive material. In either embodiment the sheath 44 is composed of a non-conductive material, and the configurations of electrically conductive energy return electrodes 14 shown in FIGS. 4 and 5 each causes a concentration of the return energy vector on the side of the sheath 44 on which the energy return electrodes 14, or the conductive portions 48 of the energy return electrodes 14, are located. When the sheath 44 is positioned such that the energy return electrodes 14 are proximate or in contact with the target tissue, delivery of the electric current from the energy delivery device 16 and flow of the electric current to the conductive portions 48 of the energy return electrodes 14 will cause the formation of a lesion in the target tissue, while minimizing or avoiding heating of the blood and potentially the formation of gas bubbles and/or char.

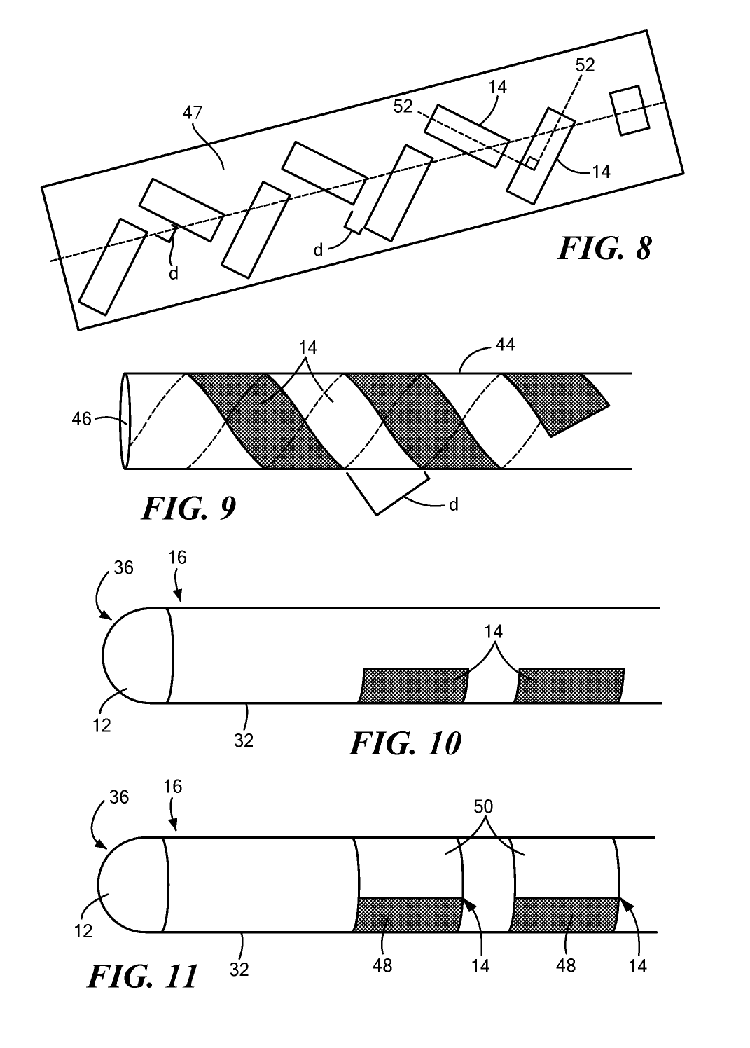

Referring to FIGS. 6-8, the sheath 44 includes a plurality of electrically conductive energy return electrodes 14, and each energy return electrode 14 extends around a portion (that is, less than an entirety) of the circumference of the sheath 44 such that adjacent energy return electrodes 14 extend around a different portion of the circumference of the sheath 44. The energy return electrodes 14 are separated by a distance d, which may be chosen according to the voltage of the pulsed field ablation energy that will be delivered by the energy delivery device 16. In one embodiment, the energy return electrodes 14 are each composed of a conductive material that is applied to (for example, adhered to, coupled to, and/or deposited on) the sheath 44. In one non-limiting example, as shown in FIG. 8, the energy return electrodes 14 may be composed of conductive material that is attached to and/or deposited onto a flexible film 47, which is then applied or mounted to the sheath 44. Additionally or alternatively, the energy return electrodes 14 are composed of a film of conductive material and applied or mounted directly to the sheath 44. In one embodiment, such as that shown in FIGS. 7 and 8, the energy return electrodes 14 are applied or mounted to the sheath 44 such that adjacent energy return electrodes 14 have orthogonal longitudinal axes 52.

Referring now to FIG. 9, in one embodiment the sheath 44 includes a spiral-shaped single energy return electrode 14 that wraps at least once around the circumference of the sheath 44. The loops of the spiral are separated by distance d.

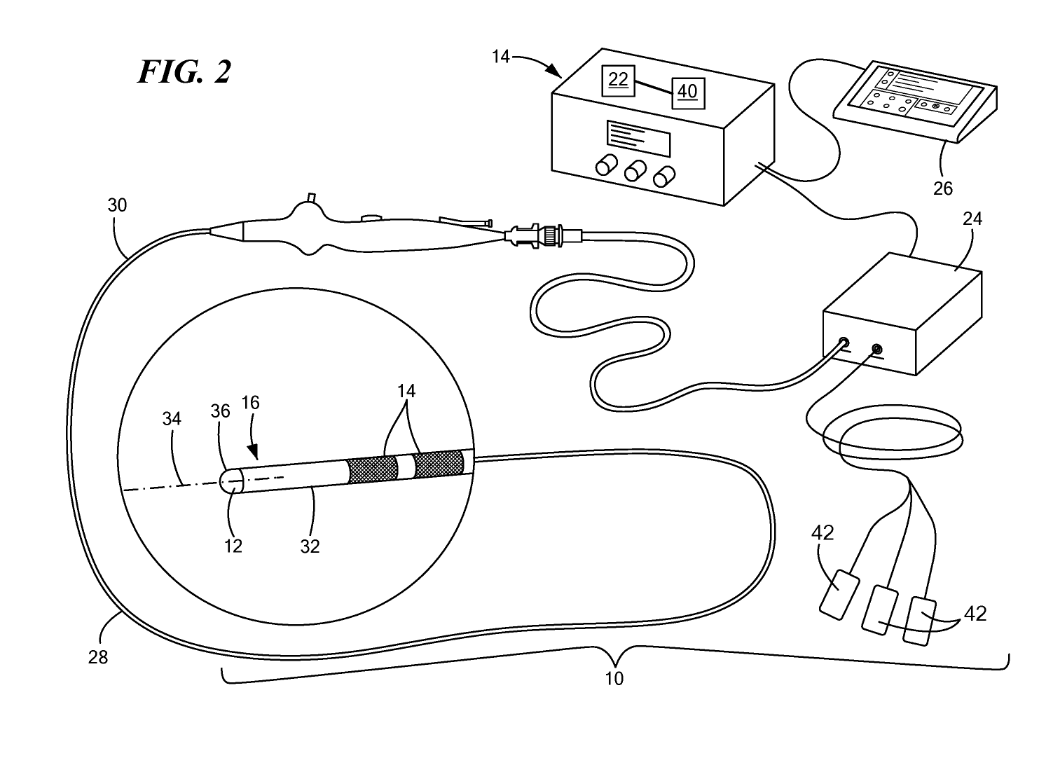

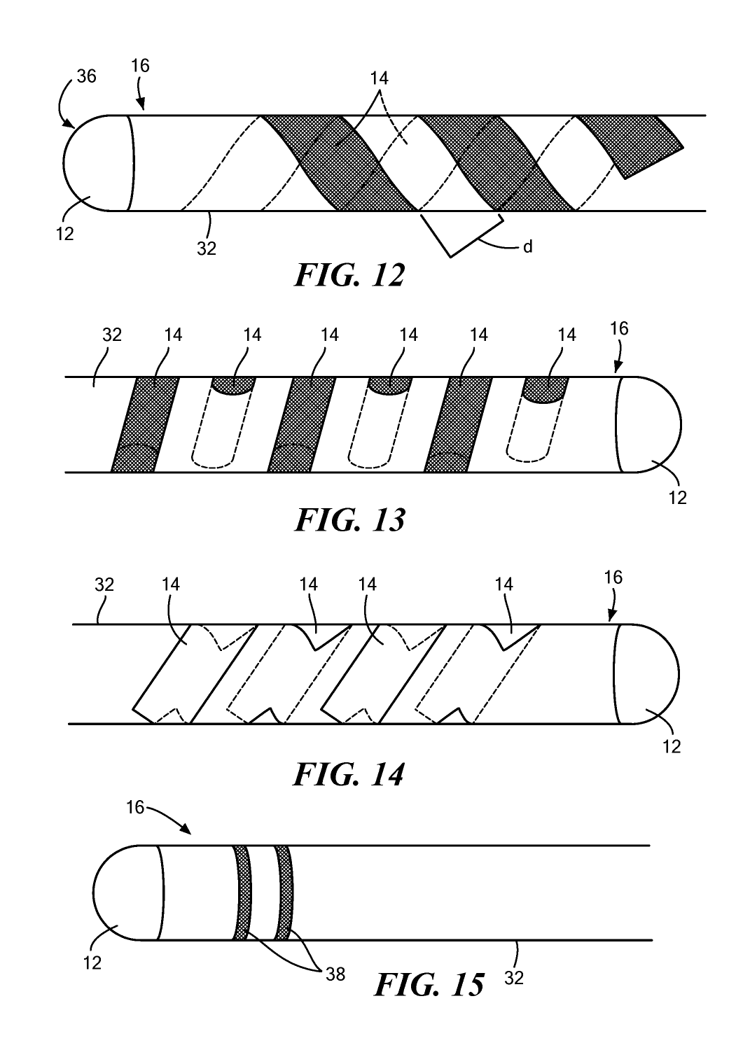

Referring now to FIG. 2, another embodiment of a medical system 10 is shown. Like the medical system 10 of FIG. 1, the medical system 10 of FIG. 2 includes an energy delivery device 16 having at least one energy return electrode 14. In one embodiment the energy delivery device 16 is be a focal catheter with an elongate body 28 and an energy delivery electrode 12 at the distal tip 36. However, other numbers and/or configurations of energy delivery electrodes 12 may be used. The energy delivery device 16 includes at least one energy return electrode 14 on the elongate body 28. As is shown in FIGS. 10-14, the at least one energy return electrode 14 may be as shown and described in FIGS. 4-9, except the energy return electrodes 14 are located on the elongate body 28 of the energy delivery device 16 rather than the sheath 44. For example, FIG. 10 shows an embodiment of the energy delivery device 16 in which the energy return electrodes 14 extend around a portion of the circumference of the elongate body 28 (compare to FIG. 4); FIG. 11 shows an embodiment of the energy delivery device 16 in which the energy return electrodes 14 extend around an entirety of the circumference of the elongate body 28, with each energy return electrode 14 having a conductive first portion 48 and a non-conductive second portion 50 (compare to FIG. 5); FIG. 12 shows embodiment of the energy delivery device 16 having a spiral-shaped single energy return electrode 14 that wraps at least once around the circumference of the elongate body 28 (compare to FIG. 9); and FIGS. 13 and 14 show an embodiment of the energy delivery device 16 having energy return electrodes 14 that each extend around a portion of the circumference of the elongate body 28 such that adjacent energy return electrodes 14 extend around a different portion of the circumference of the elongate body 28 (compare to FIGS. 6 and 7). Although the energy return electrode(s) 14 are not movable relative to the energy delivery electrode(s) 12 as in embodiments wherein the energy return electrode(s) 14 are on a sheath 44, inclusion of the energy return electrode(s) 14 on the energy delivery device 16 may simplify the treatment procedure by requiring only a single device.

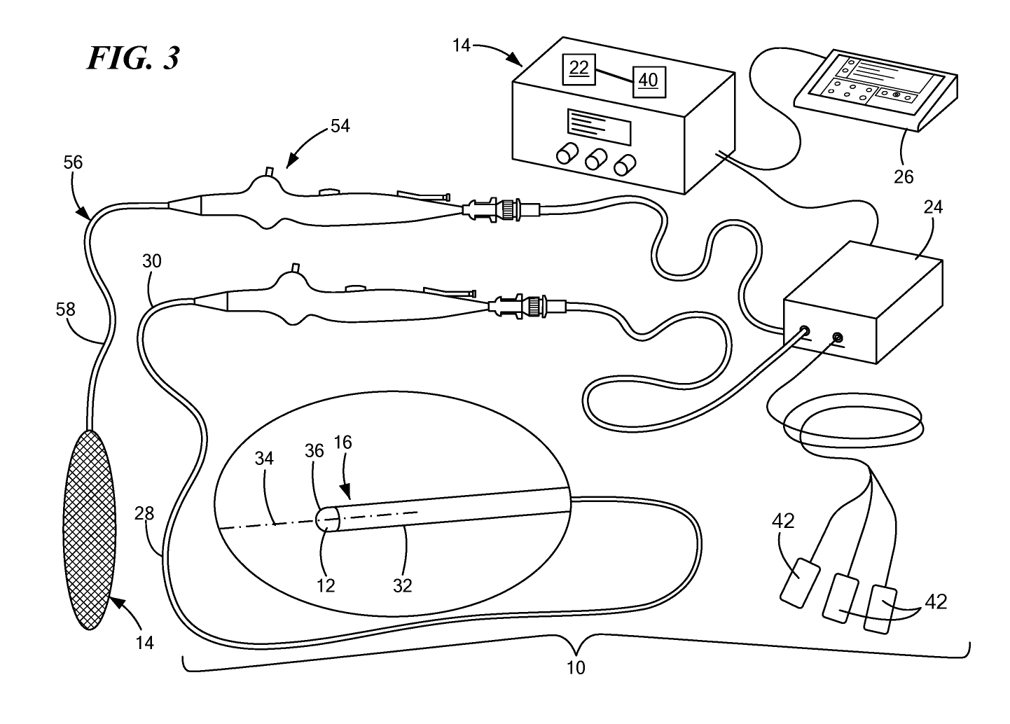

Referring now to FIG. 3, another embodiment of a medical system 10 is shown. The medical system 10 of FIG. 3 includes an energy delivery device 16 and a secondary device 54 having at least one energy return electrode 14. In one embodiment, the secondary device 54 includes an elongate body 56 and a single energy return electrode 14 at the distal portion 58 of the elongate body 56. The energy return electrode 14 has a larger surface area than the energy delivery electrode(s) 12 and is positionable independently of the energy delivery device 16, and energy may be delivered from the energy delivery electrode(s) at a first location and the energy return electrode 14 at a second location. In one embodiment, the energy delivery electrode(s) 12 of the energy delivery device 16 are positioned at an endocardial first location, such as within a cardiac vein, within a cardiac artery, in contact with tissue surrounding a pulmonary vein ostium, within or in contact with the superior or inferior vena cava, within or in contact with an appendage (such as the left atrial appendage or the right atrial appendage), within the right or left atrium (for example, in contact with atrial tissue), within the right or left ventricle (for example, in contact with ventricular tissue), within the coronary sinus, within the aorta, within the pulmonary artery, within a pulmonary vein, or the like. In another embodiment, the energy delivery electrode(s) 12 of the energy delivery device 16 are positioned at an epicardial first location, such as within the pericardial space, at a location outside but adjacent the pericardium, in contact with atrial epicardial tissue, in contact with ventricular epicardial tissue, or the like. Further, in one embodiment, the energy return electrode 14 is positioned at an endocardial or epicardial second location, such as within a cardiac vein, within a cardiac artery, in contact with tissue surrounding a pulmonary vein ostium, within or in contact with the superior or inferior vena cava, within or in contact with an appendage (such as the left atrial appendage or the right atrial appendage), within the right or left atrium (for example, in contact with atrial tissue), within the right or left ventricle (for example, in contact with ventricular tissue), within the coronary sinus, within the aorta, within the pulmonary artery, within a pulmonary vein, within the pericardial space, at a location outside but adjacent the pericardium, in contact with atrial epicardial tissue, in contact with ventricular epicardial tissue, or the like. For example, the energy delivery electrode(s) 12 of the energy delivery device 16 may be positioned within the left atrium and the energy return electrode(s) 14 of the secondary device 54 may be positioned in the pericardial space, in the superior or inferior vena cava, or the like. As another example, the energy delivery electrode(s) 12 of the energy delivery device 16 may be positioned within the pericardial space, the superior or inferior vena cava, or the like, and the energy return electrode(s) 14 of the secondary device 54 may be positioned at an ostium of a pulmonary vein, at an antrum or an area of the atrial endocardium surrounding or about a pulmonary vein ostium, within the left atrium, in contact with the right atrial endocardium or the left atrial endocardium, at a lumen of an atrial appendage (and in contact with at least a portion of tissue surrounding the lumen and/or opening), or the like. Activation of the energy delivery device 16 will transmit an electrical current between the energy delivery electrode(s) 12 and the energy return electrode(s) 14 to create transmural lesion(s) within the tissue between the energy delivery electrode(s) 12 and the energy return electrode(s) 14.

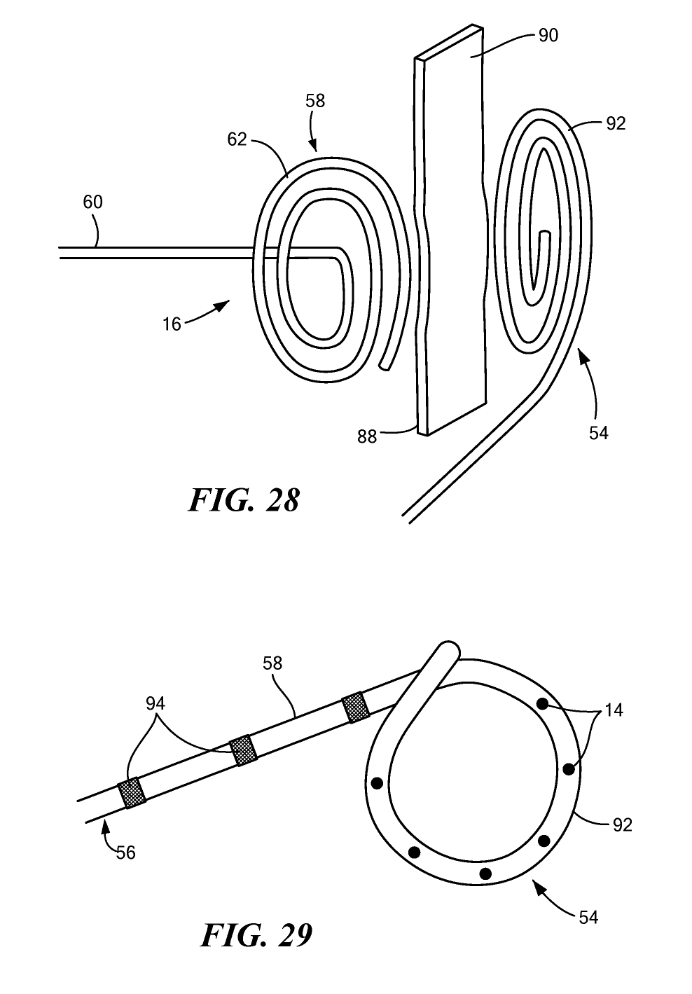

Continuing to refer to FIG. 3, in one embodiment the energy delivery device 16 is a focal catheter having an energy delivery electrode 12 at the distal tip 36 of the elongate body 28. However, referring now to FIGS. 16 and 17, the medical system 10 may include other embodiments of the energy delivery device 16. For example, in one embodiment shown in FIG. 16, the energy delivery device 16 includes a needle-shaped energy delivery electrode 12 at or extending from the distal portion 32 of the elongate body 28, such as extending from the distal tip 36 along the longitudinal axis 34 of the energy delivery device 16. Referring now to FIG. 17, in another embodiment the energy delivery device 16 includes a balloon or expandable element 64, such as a basket or mesh, coupled to the distal portion 32 of the elongate body 28 and including at least one energy delivery electrode 12, provided the energy delivery electrode(s) 12 have a smaller surface area, or energy delivery surface area, than the surface area, or energy return surface area, of the energy return electrode(s) 14. However, it will be understood that other configurations may also be used. For example, in one embodiment the energy delivery device 16 is a spiral catheter having a shaft 60 and a spiral-shaped carrier arm 62 bearing at least one energy delivery electrode 12 (for example, as shown in FIG. 28).

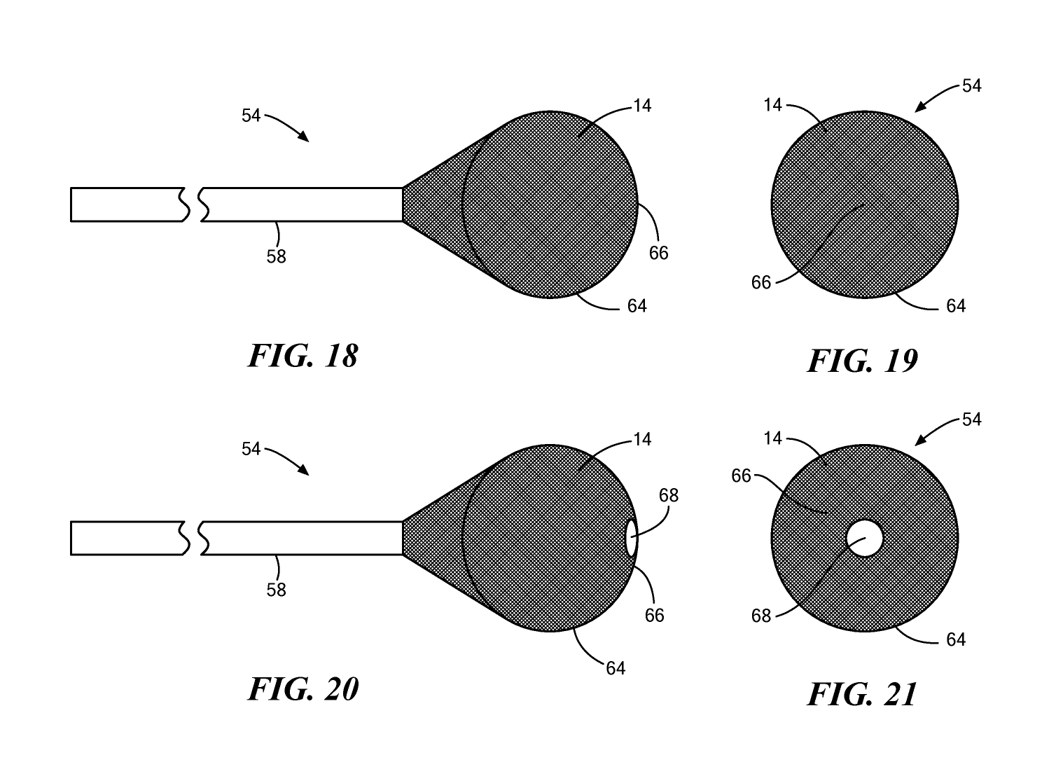

Referring now to FIGS. 18-40, embodiments of the secondary device 54 are shown. In one embodiment, as shown in FIGS. 18-23, the secondary device 54 includes a balloon or expandable element 64 (such as a basket or mesh) having at least one energy return electrode 14. For example, the secondary device 54 may include an expandable balloon 64 that is covered with mesh that includes at least one energy return electrode 14. Further, the mesh may include one or more independently controllable energy return electrode(s) 14 and/or pluralities of energy return electrode(s) 14 or conductive areas. Additionally or alternatively, the secondary device 54 may include an expandable balloon to which at least one energy return electrode 14 has been deposited, affixed, applied, adhered, or otherwise coupled. Further, each energy return electrode 14, or pluralities/groups of energy return electrodes 14, may be independently controllable.

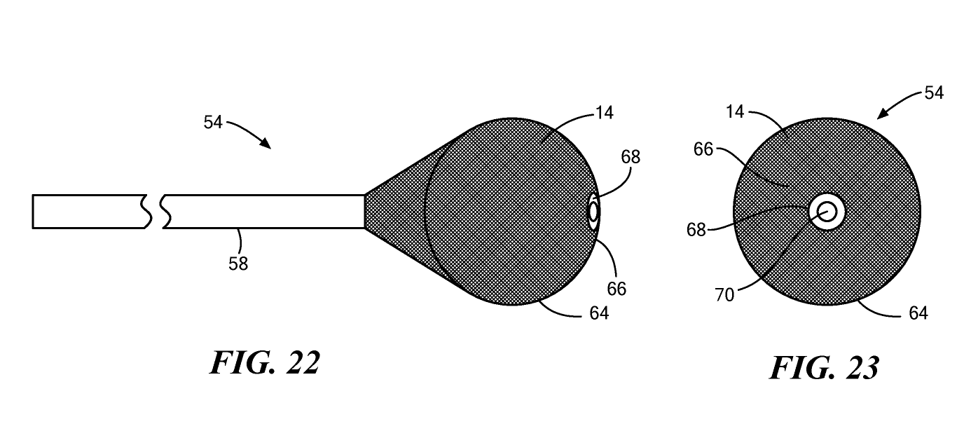

Referring to FIGS. 18 and 19, a side view and a front view, respectively, of an embodiment of a secondary device 54 are shown. The expandable element 64 of the secondary device 54 has an atraumatic distal face 66. For example, the distal face 66 may define a continuous distal surface as shown in FIGS. 18 and 19. Referring to FIGS. 20 and 21, in another embodiment the secondary device 54 includes a shaft 68 that is at least partially exposed from the expandable element 64. However, the distal tip of the shaft 68 is continuous with the distal face 66 of the expandable element 64, such that the shaft 68 does not extend beyond the distal face 66 of the expandable element 64. Referring to FIGS. 22 and 23, in one embodiment, the secondary device 54 includes a shaft 68 with a lumen 70, such as a guidewire lumen. Like the secondary device 54 shown in FIGS. 20 and 21, the secondary device 54 shown in FIGS. 22 and 23 includes a shaft 68 with a distal tip that is continuous with the distal face 66 of the expandable element 64 to create an atraumatic distal face 66.

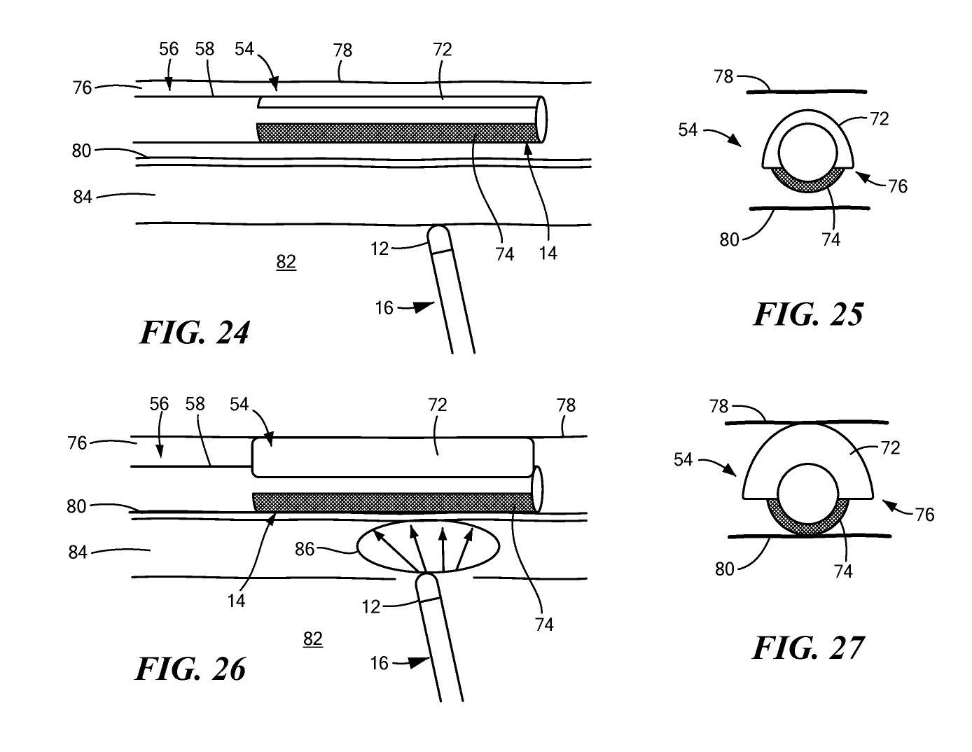

Referring now to FIGS. 24-27, in another embodiment the secondary device 54 includes an elongate body 56 having a distal portion 58, an expandable element 72 on a first side of the distal portion 58 of the elongate body 56, and at least one energy return electrode 14 on a second side of the distal portion 58, opposite the expandable element 72. Put another way, the expandable element 72 and the at least one energy return electrode 14 are on opposite sides of the elongate body 56. In one embodiment, the at least one energy return electrode 14 includes a conductive mesh 74. Additionally or alternatively, the at least one energy return electrode 14 may include a single energy return electrode 14 or a plurality of energy return electrodes 14 coupled to the elongate body 56. The expandable element 72 may include a balloon (for example, as shown in FIGS. 24-27), at least one spline, an expandable basket, or the like.

Continuing to refer to FIGS. 24-27, in one exemplary method of use, the distal portion 58 of the secondary device 54 is inserted into the pericardial space 76 with the expandable element 72 in an uninflated/unexpanded configuration (as shown in FIGS. 24 and 25). The expandable element 72 is then inflated/expanded, such as by the delivery of an inflation fluid into the expandable element 72. An exemplary expanded configuration is shown in FIGS. 26 and 27. Inflation/expansion of the expandable element 72 causes the distal portion 58 of the secondary device 54 to expand between the pericardium 78 and the epicardium 80, thereby urging the at least one energy return electrode 14 into contact, or more forceful/stable contact, with the epicardial tissue 80. An energy delivery device 16 may be positioned in, for example, the left atrium 82 of the heart. Pulsed field ablation energy delivered from the energy delivery electrode(s) 12 flows to through the myocardial tissue 84 to the energy return electrode(s) 14, thereby creating a lesion 86 in the myocardial tissue 84 between the energy delivery electrode(s) 12 and the energy return electrode(s) 14.

Referring to FIGS. 28-31, in one embodiment the secondary device 54 is a pigtail-shaped device having at least one energy return electrode 14. Further, in the embodiment shown in FIG. 28, the energy delivery device 16 is a device having a distal portion 58 in the shape of or coupled to a spiral-shaped carrier arm 62 coupled to or integrated with a shaft 60, the carrier arm 62 bearing at least one energy return electrode 14. Alternatively, the distal portion 58 may be a spiral-shaped energy delivery electrode 12. The energy return electrode(s) 14 and energy delivery electrode(s) 12 are not shown in FIG. 28 for simplicity. In use, the energy delivery electrode(s) 12 of the energy delivery device 16 are positioned proximate or in contact with a first side 88 of an area of tissue and the energy return electrode(s) 14 of the secondary device 54 are positioned proximate or in contact with a second side 90 of the area of tissue. Pulsed field ablation energy delivered from the energy delivery electrode(s) 12 flows through the tissue to the energy return electrode(s) 14 to create a lesion in the tissue.

Referring to FIG. 29, in one embodiment the secondary device 54 has an elongate body 56 with a distal portion 58 that is maneuverable into a spiral shape. The secondary device 54 shown in, for example, FIG. 29, may be referred to as a pigtail catheter, and may be preferred for use within the pericardial space 76 because of its flat profile. For example, the secondary device 54 may be delivered to a treatment site in a linear first configuration and then transitioned into a spiral-shaped, expanded second configuration. In one embodiment, the energy return electrode(s) 14 are located on the portion of the distal portion 58 of the elongate body 56 that forms the spiral or loop 92 when the elongate body 56 is in the second configuration. The energy return electrode(s) 14 may be affixed to, coupled to, adhered to, deposited onto, or otherwise coupled to the elongate body 56 on a surface that is in contact with, or closer to, the target tissue when the secondary device 54 is in use. Put another way, the energy return electrode(s) 14 may be coplanar, or at least substantially coplanar, when the secondary device 54 is in the second spiral-shaped configuration. For example, the energy return electrode(s) 14 may be located on the side of the elongate body 56 that is closer to the myocardial tissue, and away from the pericardium when the secondary device 54 is in use, which may minimize the likelihood of collateral damage to non-target structures. The secondary device 54 may also include one or more mapping electrodes 94 on the elongate body 56 proximal to the energy return electrode(s) 14 to record electrograms and/or other signals from an area of tissue. Each of the energy return electrode(s) 14 and, if included, the mapping electrode(s) 94, or groups of the electrode(s) 14, 94, may be in electrical communication with and selectively operable by the CEDS 24. Although the elongate body 56 is referred to as being transitioned into a spiral-shaped second configuration, it will be understood that the elongate body 56 may be transitioned into any suitable expanded second configuration, such as, but not limited to, a loop-shaped, curvilinear, spiral, helical, or arcuate second configuration. Further, it will be understood that the secondary device 54 may include more or fewer energy return electrode(s) 14 than those shown in FIG. 29.

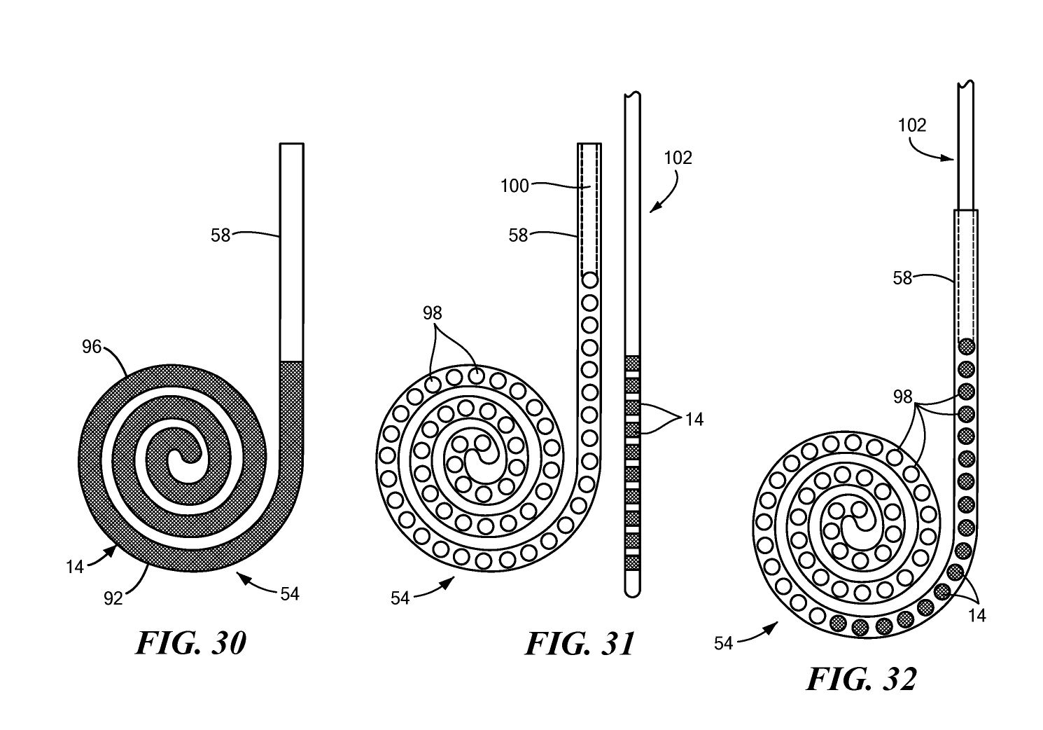

Referring to FIGS. 30-32, in one embodiment the secondary device 54 includes a first (non-tissue-contacting) surface of the secondary device 54, which is shown in FIG. 30, and a second, or tissue-contacting surface, which is shown in FIGS. 31 and 32. In one embodiment, the distal portion 58 of the secondary device 54 is transitionable into a spiral-shaped second configuration (for example, as shown in FIG. 29). In one embodiment, the first surface, or that surface configured to be oriented away from the epicardial surface (or other tissue surface) when the secondary device 54 is in use, includes or is composed of a non-conductive material 96. The non-conductive surface shown in FIG. 30 may be included in either of the secondary devices 54 shown in FIGS. 29, 31, and 32.

Referring to FIGS. 31 and 32, an embodiment of a second, or tissue-contacting, surface of the secondary device 54 shown. In one embodiment, the secondary device 54 is similar to that shown in FIGS. 29, except the energy return electrode(s) 14 are not coupled to the elongate body 56. Instead, the distal portion 58 of the elongate body 56 includes at least one aperture 98 that extends from the outer surface of the elongate body 56 to an inner lumen 100 of the elongate body 56. Like the energy return electrode(s) 14 of the secondary device 54 shown in FIG. 29, the at least one aperture 98 is located on the side of the elongate body 56 that is in contact with, or closer to, the target tissue when the secondary device 54 is in use. For example, in one embodiment the distal portion 58 of the elongate body 56 has a tissue-contacting surface when the distal portion 58 is in the spiral-shaped second configuration (for example, the tissue-contacting surface may lie in a plane, or may lie in a plane parallel to a plane, in which the spiral-shaped second configuration lies). Additionally or alternatively, the apertures 98 may be radially arranged about the distal portion 58 of the elongate body 56. The secondary device 54 also includes an energy return stylet 102 that is insertable or translatable into the inner lumen 100 of the elongate body 56 such that at least a portion of the energy return stylet 102 is exposed through the at least one aperture 98 in the distal portion 58 of the elongate body 56.

Continuing to refer to FIGS. 31 and 32, in one embodiment the energy return stylet 102 includes a plurality of energy return electrodes 14 that are alignable with the apertures 98. For example, the energy return stylet 102 may include the same number of energy return electrodes 14 as apertures 98 in the elongate body 56. Alternatively, the energy return stylet 102 may include fewer energy return electrodes 14 than apertures 98. At least a portion of the energy return stylet 102, such as at least a portion of the distal portion of the energy return stylet 102, is flexible and configured to be transitioned between the first and second configurations when inside the secondary device elongate body 56. When the energy return stylet is within the elongate body 56, the energy return electrode(s) 14 are exposed through the apertures 98. The energy return stylet 102 may be removably inserted into the secondary device elongate body 56 (for example, through the handle or proximal portion of the elongate body) or may be permanently enclosed within the secondary device elongate body 56. The energy return electrode(s) 14 may be used as mapping electrodes to record electrograms from a wide area of tissue and/or to provide an energy return path from the energy delivery device 16. Further, each of the energy return electrode(s) 14 or groups of the electrode(s) 14 may be in electrical communication with and selectively operable by the CEDS 24.

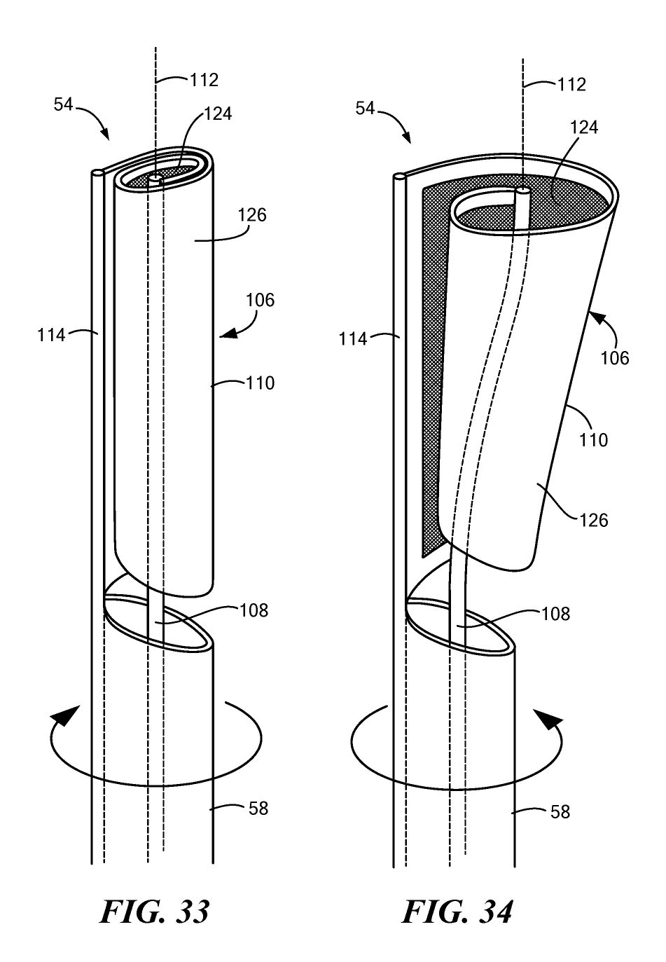

Referring now to FIGS. 33-37, embodiments of a secondary device 54 for use within a vessel are shown. Referring to FIGS. 33 and 34, in one embodiment the secondary device 54 includes an elongate body 56 having a distal portion 58 with an expandable element 106 and a shaft 108 that extends through the elongate body 56 (for example, through a central lumen) and is coupled to a portion of the expandable element 106 at the distal portion 58 of the shaft 108. The expandable element 106 includes a sheet 110 that is wound at least partially about the shaft 108, such that rotation of the shaft 108 about its longitudinal axis 112 in a first direction causes the sheet 110 to be more tightly wound about the shaft 108, thereby decreasing the outer diameter of the expandable element 106. Likewise, rotation of the shaft 108 about its longitudinal axis 112 in a second direction causes the sheet 110 to be more loosely wound about the shaft 108, thereby increasing the outer diameter of the expandable element 106. Thus, the sheet 110 may be referred to as a rolled sheet 110. In one embodiment, the secondary device 54 also includes a rod 114 within or coupled to one side of the elongate body 56 to connect the expandable element 106 to the portion of the elongate body 56 that is proximal to the expandable element 106 and to provide support and stabilization to the elongate body 56 during unrolling/expansion of the expandable element 106.

Continuing to refer to FIGS. 33 and 34, in one embodiment the sheet 110 includes a first surface 124 that is electrically conductive and serves as the energy return electrode(s) 14 and a second surface 126 opposite the first surface 124 that is electrically insulated. The sheet 110 is wound about the shaft 108 such that the electrically insulated second surface 126 is on the outside of the expandable element 106 (that is, is the surface that is configured to contact tissue) and the electrically conductive first surface 124 is on the inside of the expandable element 106 (that is, protected from contact with tissue). Rotation of the shaft 108 to expand the expandable element 106 not only facilitates flow of pulsed field ablation energy into the expandable element 106 and into contact with the electrically conductive first surface 124, but also allows the expandable element 106 to expand within a vessel, such as a pulmonary vein or vena cava, until the electrically insulated second surface 126 is in contact with the inner walls of the vessel. This enhances energy flow from the energy delivery electrode(s) 12 and, therefore, lesion creation within the target tissue.

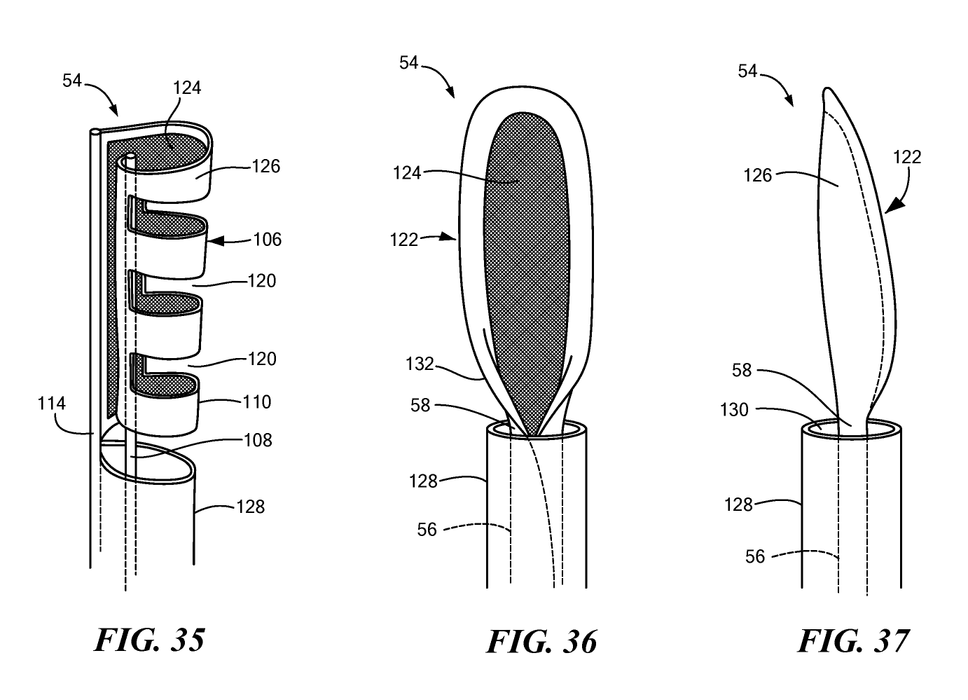

Referring to FIG. 35, in one embodiment at least a portion of the sheet 110 includes at least one slit or aperture 120, which provides additional surface area for receipt of energy from the energy delivery electrode(s) 12.

Referring to FIGS. 36 and 37, in one embodiment the secondary device 54 includes an elongate body 56 having a distal portion 58 with an expandable element 122. In one embodiment, the expandable element 122 is simply a portion of the distal portion 58 of the elongate body 56 that is slit and flattened. The distal portion 58 of the elongate body 56 includes an electrically conductive inner first surface 124 (such as a surface surrounding a central lumen within the elongate body 56) and an electrically insulated outer second surface 126. Thus, where the elongate body 56 is opened to create the expandable element 122, the expandable element 122 includes an electrically conductive first surface 124 and an electrically insulated second surface 126 opposite the first surface 124.

Continuing to refer to FIGS. 36 and 37, in an exemplary method of use the secondary device 54 is positioned at the treatment site by passing the secondary device 54 through a guide sheath 128. When in the sheath 128, the expandable element 122 is configured to be in a rolled first configuration for delivery. Once the secondary device 54 is advanced through the distal opening 130 of the sheath 128, the portion of the elongate body 56 that forms the expandable element 122 is allowed to expand and become flattened (that is, to transition to an expanded second configuration). The transition area 132 between the elongate body 56 and the expandable element 122 is sloped to facilitate recapture of the secondary device 54 within the sheath 128 for removal of the secondary device 54 from the patient's body.

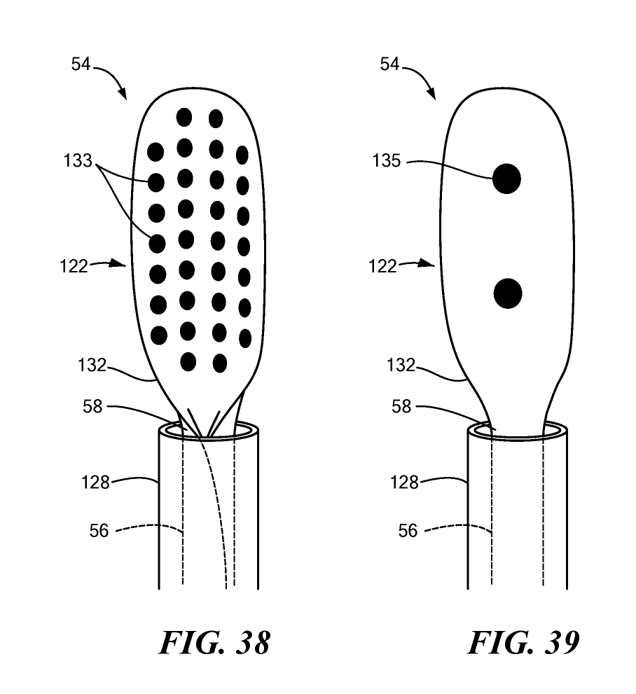

Referring now to FIGS. 38 and 39, in one embodiment the secondary device 54 includes an elongate body 56 having a distal portion 58 with an expandable element 122. In one embodiment, the secondary device 54 shown in FIG. 38 is similar to the secondary device 54 shown in FIGS. 36 and 37. Thus, in one embodiment, the expandable element 122 is simply a portion of the distal portion 58 of the elongate body 56 that is slit and flattened. However, in contrast to the secondary device 54 shown in FIGS. 36 and 37, in one embodiment the distal portion 58 of the elongate body 56 of the secondary device 54 of FIG. 38 includes a plurality of electrically conductive elements 133 on the inner first surface 124 (such as a surface surrounding a central lumen within the elongate body 56) and an electrically insulated outer second surface 126. Thus, wherein the elongate body 56 is opened to create the expandable element 122, the expandable element 122 includes a plurality of electrically conductive elements 133 (for example, electrodes) on the first surface 124 and an electrically insulated second surface 126 opposite the first surface 124. The electrically conductive elements 133 may be flat or flush with the inner first surface 124 or they may each have a raised profile forming a matrix of prominent electrodes.

Continuing to refer to FIGS. 38 and 39, in an exemplary method of use the secondary device 54 is positioned at the treatment site by passing the secondary device 54 through a guide sheath 128. When inside the guide sheath 128, the expandable element 122 is configured to be rolled in a rolled first configuration for delivery. Once the secondary device 54 is advanced through the distal opening 130 of the sheath 128, the portion of the elongate body 56 that forms the expandable element 122 is allowed to expand and become flattened (that is, to transition to an expanded second configuration). In one embodiment, the transition area 132 between the elongate body 56 and the expandable element 122 is sloped to facilitate recapture of the secondary device 54 within the guide sheath 128 for removal of the secondary device 54 from the patient's body. An exemplary placement of the plurality of electrically conductive elements 133 is on the epicardial surface of the right ventricular outflow tract. In one embodiment, the electrically conductive elements 133 have a raised profile and may be used to collect signals, such as monophasic action potential signals, from the tissue with which the electrically conductive elements 133 are in contact to evaluate the condition of the underlying cardiomyocytes. For such use, in one embodiment one or more reference electrodes 135 may be located on the second surface 126 of the secondary device 54. A non-limiting example of reference electrodes 135 is shown in FIG. 39. Further, the electrically conductive elements 133 may be selectively activated such that the secondary device 54 may be used to deliver pulsed electric fields through selected ones of the plurality of electrically conductive elements 133.

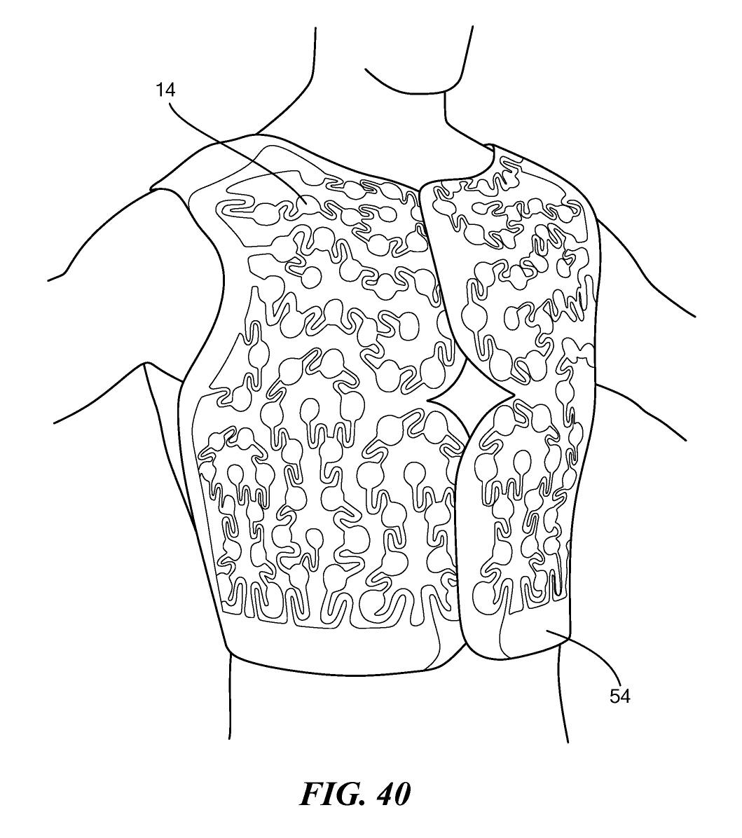

Referring now to FIG. 40, in one embodiment the secondary device 54 is a vest worm by the patient that includes many energy return electrodes 14 (for example, 252 energy return electrodes 14) that serve as the electrical return path for pulsed field ablation energy deliveries, such that the body surface electrode area is so great that the electric field gradient is below the threshold for muscle and nerve stimulation. Thus, a focal energy delivery device 16 may be used to ablate the targeted tissue within the body and the large energy return electrode surface area provided by the vest may provide the energy return path.

In all embodiments of the sheath 44, energy delivery device 16, and secondary device 54 having at least one energy return electrode 14, each energy return electrode 14, or portion of each energy return electrode 14, may be independently controllable by the control unit 18 and/or CEDS 24. For example, each energy return electrode 14 or portion thereof may be selectively activated or deactivated for selective site conductivity and/or electrogram recording.

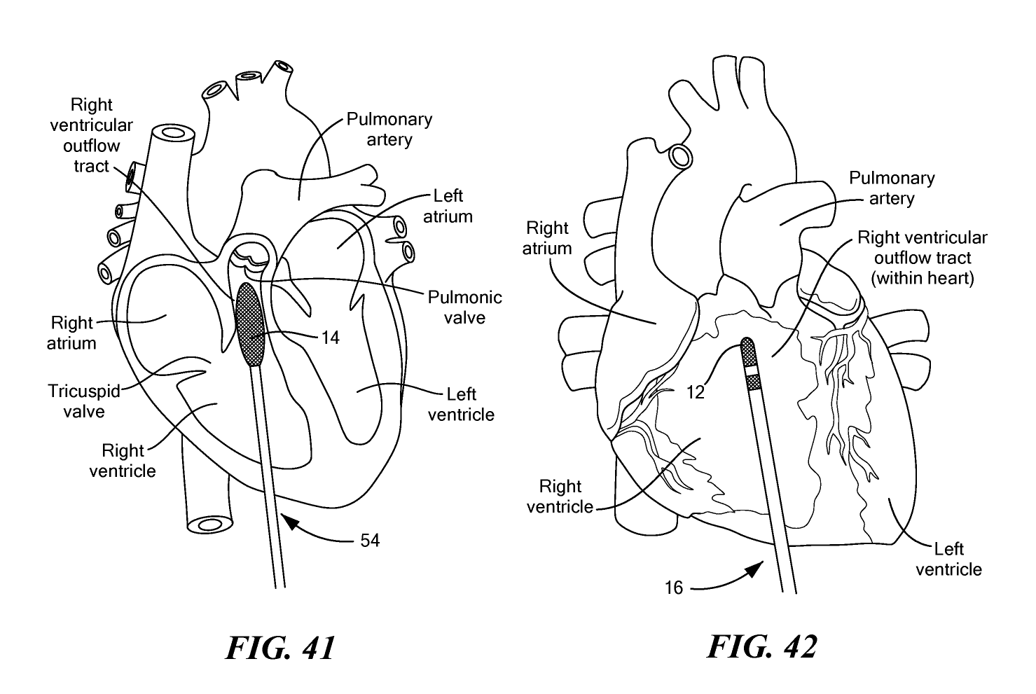

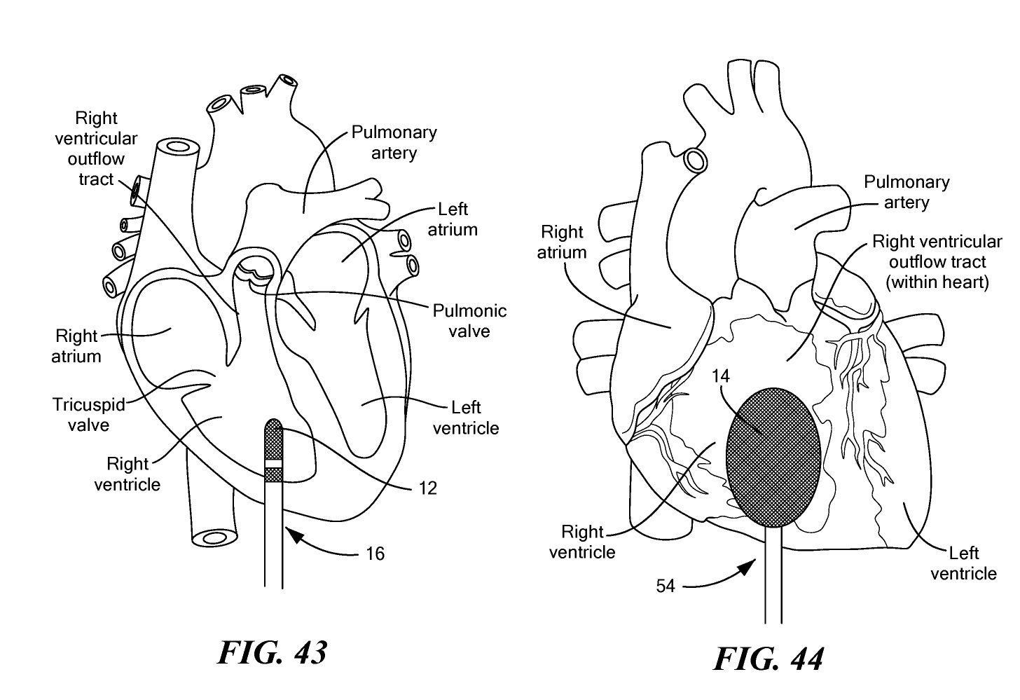

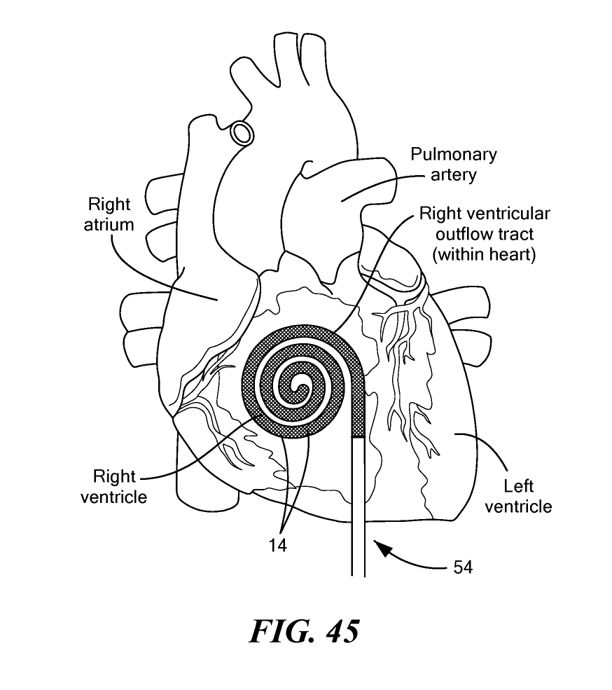

Referring now to FIGS. 41-45, exemplary placements of the energy delivery device 16 and secondary device 54 during a treatment procedure are shown. Referring to FIGS. 41 and 42, in one non-limiting example, the secondary device 54 is positioned within the heart, such as within the right ventricular outflow tract (for example, as shown in FIG. 41), and the energy delivery device 16 is positioned in contact with the epicardium, proximate the location of the secondary device 54 (for example, as shown in FIG. 42). Pulsed field ablation energy delivered by the energy delivery electrode(s) 12 of the energy delivery device 16 flows through the myocardial tissue to energy return electrode(s) 14 of the secondary device 54. Referring to FIGS. 43-45, in another non-limiting example, the energy delivery device 16 is positioned within the heart, such as within the right ventricle (for example, as shown in FIG. 43), and the secondary device 54 is positioned in contact with the epicardium, proximate the location of the energy delivery device 16. Pulsed field ablation energy delivered by the energy delivery electrode(s) 12 of the energy delivery device 16 flows through the myocardial tissue to energy return electrode(s) 14 of the secondary device 54. The secondary device 54 may have any suitable configuration and/or size/number of energy return electrode(s) 14. In one embodiment, as shown in FIG. 44, the secondary device 54 includes an expandable element with one or more energy return electrode(s) 14, such as the expandable element 64 shown in FIGS. 18-23. In another embodiment, as shown in FIG. 45, the secondary device 54 includes a distal portion transitionable to a spiral-shaped expanded configuration, such as the secondary device shown in FIGS. 28-32.

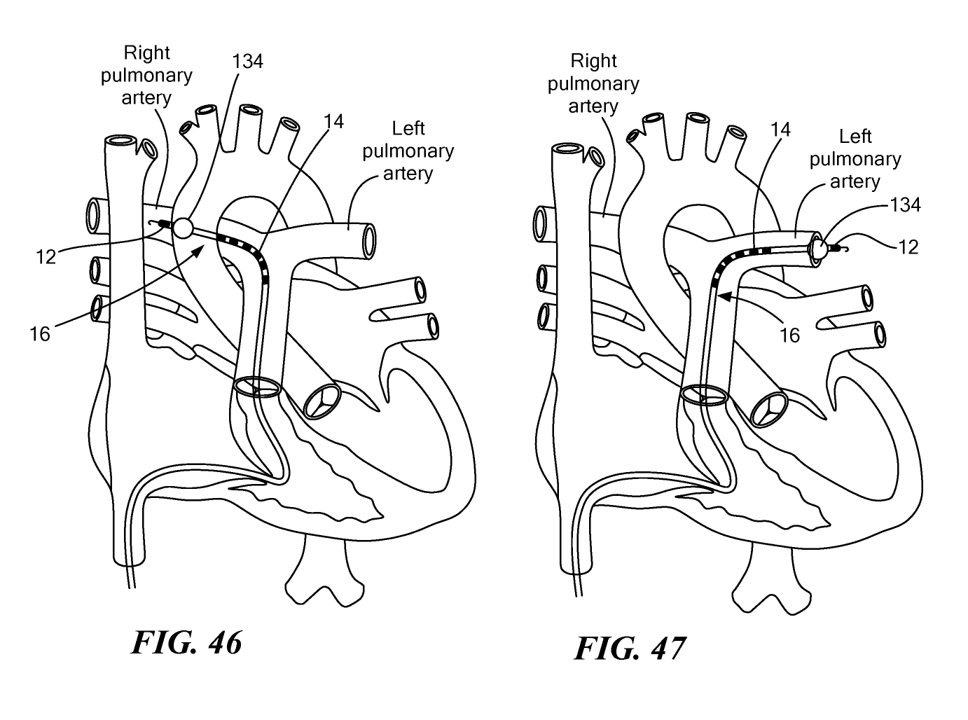

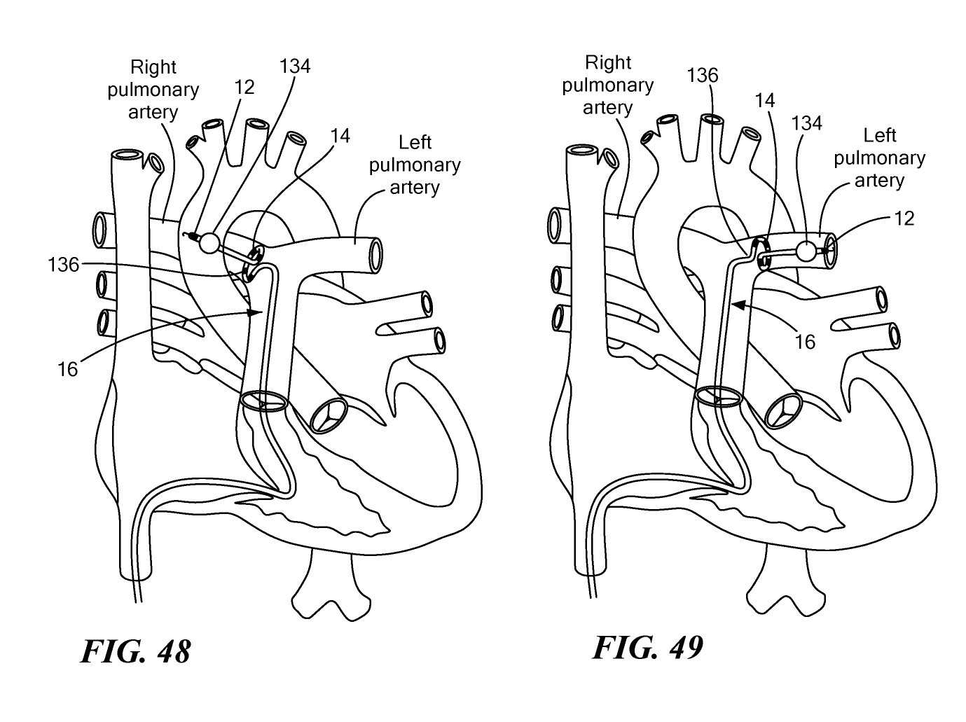

Referring now to FIGS. 46-49, exemplary placements of an energy delivery device 16 are shown, the energy delivery device including at least one energy return electrode 14. In the non-limiting examples shown, the energy delivery device 16 includes a distal tip energy delivery electrode 12, at least one energy return electrode 14 on the elongate body distal portion 32 proximal to the energy delivery electrode 12, and a balloon or expandable element 134 to facilitate placement of the energy delivery device at the treatment site. In one non-limiting example, the energy delivery device 16 is positioned such that the energy delivery electrode 12 and balloon 134 are positioned within the right pulmonary artery (for example, as shown in FIGS. 46 and 48). Alternatively, the energy delivery device 16 may be positioned such that the energy delivery electrode 12 and balloon 134 are positioned within the left pulmonary artery (for example, as shown in FIGS. 47 and 49). In one embodiment, the elongate body distal portion 32 is linear, or at least substantially linear (for example, as shown in FIGS. 46 and 47). In another embodiment, the elongate body distal portion 32 is transitionable to an expanded configuration including a loop portion 136 that bears the energy return electrode(s) 14. However, it will be understood that the energy delivery device 16 may have any suitable size, shape, or configuration that provides a sufficient energy return electrode surface area.

Regardless of the configuration of the energy delivery device 16 and/or the secondary device 54, it will be understood that either device 16, 54 may operate as either an energy delivery device or secondary (energy return) device. Similarly, each electrically conductive component of a single device may include one or more selectively activatable electrodes (for example, a device may include one or more energy delivery electrodes 12 and one or more energy return electrodes 14, including electrode(s) 12, 14 on the elongate body 28 and/or a sheath 44, as discussed above). That is, each device 16 and/or 54, and/or components thereof, may be operable to deliver energy and/or to serve as a return device. In one embodiment, the energy delivery device 16 includes a plurality of electrodes 12 and the energy return device 54 includes a plurality of electrodes 14. If fewer electrodes 12 (or electrodes representing a smaller surface area) are selectively activated on the energy delivery device 16 than electrodes 14 selectively activated on the secondary device 54 (or electrodes representing a larger surface area), an energy vector will be created from the electrode(s) 12 on the energy delivery device 16 to the return electrode(s) 14 on the secondary device 54. Conversely, a reverse energy vector may be created if more electrodes 12 (or electrodes representing a larger surface area) are selectively activated on the energy delivery device 16 than electrodes 14 selectively activated on the secondary device 54 (or electrodes representing a smaller surface area), thereby causing the secondary device 54 to function as an energy delivery device and the energy delivery device 16 to function as a secondary or energy return device. Thus, in some embodiments, two identical devices may be used in a procedure (within both devices being in communication with the energy generator) and the energy vectors applied to targeted tissue may be reversed during the procedure to more efficiently ablate the targeted tissue.

### Embodiments

In one embodiment, a system for ablating tissue comprises: at least one energy delivery electrode having a first surface area; an energy generator in electrical communication with the at least one energy delivery electrode and being configured to transmit an electrical current to the at least one energy delivery electrode; and at least one energy return electrode having a second surface area that is greater than the first surface area, the at least one energy return electrode being in electrical communication with the at least one energy delivery electrode, such that electrical current delivered from the at least one energy delivery electrode to an area of tissue flows to the at least one energy return electrode.

In one aspect of the embodiment, the electrical current is pulsed field ablation energy.

In one aspect of the embodiment, the system further comprises an energy delivery device, the at least one energy delivery electrode being on the energy delivery device.

In one aspect of the embodiment, the energy delivery device includes an elongate body having a distal portion, the at least one energy return electrode being on the distal portion of the elongate body at a location that is proximal to the at least one energy delivery electrode.

In one aspect of the embodiment, the at least one energy return electrode includes a plurality of electrodes that each extends at least partially around a circumference of the elongate body. In one aspect of the embodiment, the distal portion of the elongate body includes a distal tip and the at least one energy delivery electrode is an energy delivery electrode located at the distal tip. In one aspect of the embodiment, the energy delivery electrode is a needle-shaped electrode.

In one aspect of the embodiment, the system further comprises a sheath, the energy delivery device being longitudinally movable within the sheath, the at least one energy return electrode being on the sheath. In one aspect of the embodiment, the at least one energy return electrode is movable relative to the at least one energy delivery electrode.

In one aspect of the embodiment, the system further comprises a secondary device, the at least one energy return electrode being on the secondary device.

In one aspect of the embodiment, the secondary device includes an expandable element having a conductive mesh.

In one aspect of the embodiment, the secondary device includes: a secondary device elongate body having a distal portion; an expandable element coupled to a first side of the distal portion of the secondary device elongate body; and a conductive portion coupled to a second side of the distal portion of the secondary device elongate body, the second side being opposite the first side, the conductive portion including the at least one energy return electrode.

In one aspect of the embodiment, wherein the at least one energy return electrode includes a plurality of electrodes and the secondary device includes a secondary device elongate body having a distal portion, the distal portion being transitionable between a linear first configuration and a spiral-shaped second configuration, the plurality of energy return electrodes being on a first side of the distal portion such that the plurality of energy return electrodes are coplanar with the distal portion is in the spiral-shaped second configuration.

In one aspect of the embodiment, the secondary device includes: a secondary device elongate body having a distal portion, the distal portion being transitionable between a linear first configuration and a spiral-shaped second configuration, the distal portion including a plurality of apertures; and an electrically conductive conductor insertable into the secondary device elongate body such that at least a portion of the conductor is exposed through the plurality of apertures, the at least one energy return electrode being the at least a portion of the conductor that is exposed through the plurality of apertures.

In one aspect of the embodiment, the secondary device includes: a secondary device elongate body having a distal portion, the distal portion being transitionable between a first configuration and an expanded second configuration; a shaft at least partially within the secondary device elongate body, the shaft including a distal portion and a longitudinal axis; an expandable element that is coupled to the distal portion of the shaft, the sheet being at least partially would about the shaft, the expandable element having an electrically conductive first surface and an electrically insulated second surface opposite the first surface, rotation of the shaft about its longitudinal axis causing the expandable element to transition between a first configuration and an expanded second configuration. In one aspect of the embodiment, the expandable element is a sheet.

In one embodiment, a method for ablating an area of tissue using pulsed field ablation energy comprises: positioning at least one energy delivery electrode at a first location proximate the area of tissue, the at least one energy delivery electrode having a first surface area; positioning at least one energy return electrode at a second location different than the first position, the at least one energy return electrode having a second surface area that is greater than the first surface area; and delivering the pulsed field ablation energy from the at least one energy delivery electrode to the area of tissue, such that the pulsed field ablation energy flows from the area of tissue to the at least one energy return electrode.

In one aspect of the embodiment, the at least one energy delivery electrode is on an energy delivery device and the at least one energy return electrode is on secondary device.

In one aspect of the embodiment, the first location is a location in contact with a first side of the area of tissue and the second location is a location in contact with a second side of the area of tissue opposite the first side.

In one aspect of the embodiment, the first location is within the pericardial space and the second location is in contact with an ostium of a pulmonary vein.

In one aspect of the embodiment, the first location is within the right ventricular outflow tract and the second location is within the pericardial space. In one aspect of the embodiment, the first location is within the pericardial space and the second location is within the right ventricular outflow tract. In one aspect of the embodiment, the first location is within the pericardial space and the second location is within the cardiac ventricle.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A system for ablating tissue, the system comprising:
at least one energy delivery electrode having a first surface area;
an energy generator in electrical communication with the at least one energy delivery electrode and being configured to transmit an electrical current to the at least one energy delivery electrode; and
at least one energy return electrode having a second surface area that is greater than the first surface area, the at least one energy return electrode being in electrical communication with the at least one energy delivery electrode, such that electrical current delivered from the at least one energy delivery electrode to an area of tissue flows to the at least one energy return electrode.

2. The system of claim 1, further comprising an energy delivery device, the at least one energy delivery electrode being on the energy delivery device.

3. The system of claim 2, wherein the energy delivery device includes an elongate body having a distal portion, the at least one energy return electrode being on the distal portion of the elongate body at a location that is proximal to the at least one energy delivery electrode.

4. The system of claim 3, wherein the at least one energy return electrode includes a plurality of electrodes that each extends at least partially around a circumference of the elongate body.

5. The system of claim 4, wherein the distal portion of the elongate body includes a distal tip and the at least one energy delivery electrode is an energy delivery electrode located at the distal tip.

6. The system of claim 5, wherein the energy delivery electrode is a needle-shaped electrode.

7. The system of claim 2, further comprising a sheath, the energy delivery device being longitudinally movable within the sheath, the at least one energy return electrode being on the sheath and the at least one energy return electrode being movable relative to the at least one energy delivery electrode.

8. The system of claim 2, further comprising a secondary device, the at least one energy return electrode being on the secondary device.

9. The system of claim 8, wherein the secondary device includes an expandable element having a conductive mesh.

10. The system of claim 9, wherein the secondary device includes:
a secondary device elongate body having a distal portion;
an expandable element coupled to a first side of the distal portion of the secondary device elongate body; and
a conductive portion coupled to a second side of the distal portion of the secondary device elongate body, the second side being opposite the first side, the conductive portion including the at least one energy return electrode.

11. The system of claim 8, wherein the at least one energy return electrode includes a plurality of electrodes and the secondary device includes a secondary device elongate body having a distal portion, the distal portion being transitionable between a linear first configuration and a spiral-shaped second configuration, the plurality of energy return electrodes being on a first side of the distal portion such that the plurality of energy return electrodes are coplanar with the distal portion is in the spiral-shaped second configuration.

12. The system of claim 8, wherein the secondary device includes:
a secondary device elongate body having a distal portion, the distal portion being transitionable between a linear first configuration and a spiral-shaped second configuration, the distal portion including a plurality of apertures; and
an electrically conductive conductor insertable into the secondary device elongate body such that at least a portion of the conductor is exposed through the plurality of apertures, the at least one energy return electrode being the at least a portion of the conductor that is exposed through the plurality of apertures.

13. The system of claim 12, wherein the plurality of apertures are radially arranged about the distal portion of the secondary device elongate body.

14. The system of claim 12, wherein the secondary device elongate body has a tissue-contacting surface when the secondary device elongate body is in the spiral-shaped second configuration, the plurality of apertures being on the tissue-contacting surface.

15. The system of claim 8, wherein the secondary device includes:
a secondary device elongate body having a distal portion, the distal portion being transitionable between a first configuration and an expanded second configuration;
a shaft at least partially within the secondary device elongate body, the shaft including a distal portion and a longitudinal axis; and
an expandable element that is coupled to the distal portion of the shaft, the expandable element being at least partially would about the shaft, the expandable element having an electrically conductive first surface and an electrically insulated second surface opposite the first surface,
rotation of the shaft about its longitudinal axis causing the expandable element to transition between a first configuration and an expanded second configuration.

16. The system of claim 15, wherein the expandable element is a sheet.

17. A method for ablating an area of tissue using pulsed field ablation energy, the method comprising:
positioning at least one energy delivery electrode at a first location proximate the area of tissue, the at least one energy delivery electrode having a first surface area;
positioning at least one energy return electrode at a second location different than the first location, the at least one energy return electrode having a second surface area that is greater than the first surface area; and
delivering the pulsed field ablation energy from the at least one energy delivery electrode to the area of tissue, such that the pulsed field ablation energy flows from the area of tissue to the at least one energy return electrode.

18. The method of claim 17, wherein the at least one energy delivery electrode is on an energy delivery device and the at least one energy return electrode is on secondary device.

19. The method of claim 18, wherein the first location is an endocardial location and the second location is an endocardial location.

20. The method of claim 19, wherein:
the first location is one of within a cardiac vein, within a cardiac artery, in contact with tissue surrounding a pulmonary vein ostium, within a superior vena cava, within an inferior vena cava, within an atrial appendage, within a right atrium, within a left atrium, within a right ventricle, within a left ventricle, within a coronary sinus, within an aorta, within a pulmonary artery, and within a pulmonary vein; and
the second location is one of within a cardiac vein, within a cardiac artery, in contact with tissue surrounding a pulmonary vein ostium, within a superior vena cava, within an inferior vena cava, within an atrial appendage, within a right atrium, within a left atrium, within a right ventricle, within a left ventricle, within a coronary sinus, within an aorta, within a pulmonary artery, and within a pulmonary vein.

21. The method of claim 18, wherein the first location is an endocardial location and the second location is an epicardial location.

22. The method of claim 21, wherein:
the first location is one of within a cardiac vein, within a cardiac artery, in contact with tissue surrounding a pulmonary vein ostium, within a superior vena cava, within an inferior vena cava, within an atrial appendage, within a right atrium, within a left atrium, within a right ventricle, within a left ventricle, within a coronary sinus, within an aorta, within a pulmonary artery, and within a pulmonary vein; and
the second location is one of within a pericardial space, at a location outside but adjacent a pericardium, in contact with atrial epicardial tissue, and in contact with ventricular epicardial tissue.

23. The method of claim 18, wherein the first location is an epicardial location and the second location is an endocardial location..

24. The method of claim 21, wherein:
the first location is one of within a pericardial space, at a location outside but adjacent a pericardium, in contact with atrial epicardial tissue, and in contact with ventricular epicardial tissue; and
the second location is one of within a cardiac vein, within a cardiac artery, in contact with tissue surrounding a pulmonary vein ostium, within a superior vena cava, within an inferior vena cava, within an atrial appendage, within a right atrium, within a left atrium, within a right ventricle, within a left ventricle, within a coronary sinus, within an aorta, within a pulmonary artery, and within a pulmonary vein.

25. A system for ablating tissue, the system comprising:
a first device having a plurality of first electrodes, each of the plurality of first electrodes being independently operable;
a second device having a plurality of second electrodes, each of the plurality of second electrodes being independently operable; and
an energy generator in electrical communication with the plurality of first electrodes and the plurality of second electrodes and being configured to selectively transmit an electrical current to each electrode of the plurality of first plurality of electrodes and to each electrode of the plurality of second electrodes,
the first device transmitting energy from the energy generator to the second device when a first number of electrodes of the plurality of first electrodes is activated that is less than a second number of electrodes of the plurality of second electrodes, and the second device transmitting energy from the energy generator to the first device when the first number of electrodes of the plurality of first electrodes is activated that is greater than a second number of electrodes of the plurality of second electrodes.
